(19) United States
(12) Patent Application Publication
(10) Pub. No.: US 2004/0131543 A1
(43) Pub. Date: Jul. 8, 2004

(54) Radiopharmaceuticals and radioactive microspheres for locoregional ablation of abnormal tissues

(76) Inventors: Franklin C. Wong, Sugarland, TX (US); Shuang Wang, Chicago, IL (US)

Correspondence Address: VINSON & ELKINS, L.L.P., 1001 FANNIN STREET, 2300 FIRST CITY TOWER, HOUSTON, TX, 77002-6760, US

(21) Appl. No.: 10/724,027

(22) Filed: Nov. 26, 2003

Related U.S. Application Data
(60) Provisional application No. 60/429,950, filed on Nov. 27, 2002.
(60) Provisional application No. 60/486,177, filed on Jul. 10, 2003.

(51) Int. Cl. A61K 51/00

(52) U.S. Cl. 424/1.11

(57) ABSTRACT

The present disclosure provides radiopharmaceutical macroaggregates for the treatment of abnormal tissue comprising particles having a minimum size of one micron, wherein the particles comprise iron, gadolinium, or calcium, and one or more radioactive isotopes, and have sufficient radioactivity for locoregional ablation of cells in the abnormal tissue. The disclosure also provides methods for using these radiopharmaceutical macroaggregates for the locoregional treatment of abnormal tissue.

### STATEMENT REGARDING FEDERALLY SPONSORED RESEARCH OR DEVELOPMENT

Not applicable.

### REFERENCE TO A “Microfiche Appendix”

Not applicable.

### BACKGROUND OF THE INVENTION

1. Field of the Invention

The present disclosure relates to radioactive compounds and methods for the preparation thereof, as well as methods for the treatment of abnormal tissues using the radioactive compounds.

2. Description of Related Art

In diagnostic nuclear medicine, radiopharmaceuticals containing a radionuclide which emits gamma radiation can be administered to a patient and the resulting distribution of radioactivity can be imaged with a gamma-detecting camera. The use of these diagnostic radionuclides began in the 1960's, and because they are used for diagnostic purposes only, these diagnostic radionuclides have only low levels of radioactivity (Colombetti et al., J Nucl Med 11: 704-707,1970; Stern et al., Nucleonics 24(10):57-59, 1966; Goodwin et al., JAMA 206:339-43, 1968; Barker and Gusmano, J Nucl Med 12:580-82, 1971; Brookeman et al., Am J Roentgenol Radium Ther Nucl Med 109:735-41, 1970; Wright F W, British J Radiology 47:64-65, 1974). High doses of radioactivity were avoided because of fears of radiotoxicity. Today, a wide variety of diagnostic radiopharmaceutical agents are available to assist in the diagnosis of many medical problems, for example cardiovascular, bone, kidney, lung, liver disease, infection, and cancer (Abrams and Murrer, Science 261:725-20, 1993).

A commonly used diagnostic radionuclide is Technetium-99m (^{99m}Tc), which is well suited for detection by a gamma camera because it emits gamma radiation without significant radiotoxic alpha or beta emissions (Bligh et al., Int J Rad Appl Instrum 40:751-57, 1989). An example of a nuclear medicine procedure that utilizes this diagnostic radionuclide is breast lymphoscintigraphy for breast cancer patients. This procedure aids in the identification of sentinel lymph node(s) before surgery by injecting the diagnostic radionuclide into the breast tissue surrounding the tumor and externally visualizing the movement of the radionuclide into the lymph nodes. Breast lymphoscintigraphy involves intra-parenchymal injection and subsequent visualization of the injected diagnostic radionuclide. Typically, aliquot(s) of about 1 cc containing 0.5 mCi of ^{99m}Tc labeled sulfur colloid (SC) is injected percutaneously into the tumor or breast tissues around the tumor. The smaller sizes (<0.22 micron) of SC allow for better lymphatic drainage and therefore better visualization of the sentinel lymph node(s). Only a small fraction (<1%) (Johnson et al., American Journal of Surgery 179:386-88, 2000; Doting et al., Cancer 88:2546-52, 2000) of the SC injected ever drains via the lymphatics to allow visualization of the sentinel lymph node(s). Larger size (>0.22 micron) or direct intratumoral (IT) injection of SC into the breast tumor reveals even less lymphatic drainage.

Although unsealed, diagnostic radionuclides injected into a tumor or surrounding tissues are subject to spatial sequestration. The injection site appears spherical and unchanged for hours on scintigrams. Although difficult to quantify, ultrasound guidance during selected breast lymphoscintigraphy shows that injections of SC into the breast tissue result in a larger dispersed volume. But radiation dosimetry of breast lymphoscintigraphy have shown variations up to ten-fold (Bergqvist et al., J Nucl Med 23:698-705, 1982; Glass et al., Ann Surg Oncol 6:10-11, 1999; Hung et al., J Nucl Med 356:1895-1901, 1995), due to the imprecision in determining the volume of the dispersed injectate. Although it has been proposed that diagnostic radionuclides may serve as a model for the evaluation of potential therapeutic radionuclides, existing dosimetry reports of these radionuclides are inaccurate because there is no way to accurately measure the volume of the dispersed injectate. Additionally, there is no consistent dosimetry model for locoregional injection.

In addition to diagnostic radiopharmaceutical agents, radiotherapeutic agents are also available for treating many medical problems. If appropriate, directed local treatment of cancer is often preferable to conventional radiation treatments, which can be accompanied by very harmful side effects for the patient. Additionally, directed local treatment of cancer may be a more effective therapeutic alternative. For example, multiple trials of breast conservation in patients treated with and without whole breast radiation have shown that the majority (>90%) of local recurrences of the cancer occur at the site of surgical resection (Vaidya and Baum, Eur J Cancer 34:1143-44, 1998). These trials suggest that conventional radiation treatment of the whole breast following breast conserving surgery is a radical and often unnecessary approach for the majority of women. Therefore, more directed local treatment with radiotherapy would be a preferable and safer therapy.

Radiotherapeutic agents include radionuclides with alpha or energetic beta emissions that can be targeted to disease sites. Optimal radiotherapeutic agents deposit sufficient radioactivity in target tissues to kill desired cells while minimizing uptake in nontarget tissues. For example, one therapeutic use of these radiotherapeutic agents is for the locoregional ablation of cancerous cells or tumors. Currently, only a few radiopharmaceuticals are available for locoregional treatment of cancerous cells or tumors, such as the recently FDA approved Y-90 particles (Sir-Spheres®) (U.S. Pat. Nos. 6,537,518, 6,258,338 and 5,885,547). But these radiopharmaceuticals cannot be accurately localized externally, nor can their distribution be accurately determined over time after injection. Thus, any use of these radiopharmaceuticals necessarily involves multiple approximations of, for example, total liver radiation absorbed dose rather than any actual or exact calculations of tumor radiation absorbed dose.

Currently, directed local treatment of cancer can be achieved by implanting sealed radiation sources into, for example, a post-surgical field for several weeks. Conventional brachytherapy involves the implantation of sealed radiation sources into the post-surgical field for several weeks (Nag et al., Oncology 15:195-202, 2001). Additionally, recent clinical trials have reported favorable outcomes for treating brain and breast cancer patients using a single implanted catheter filled with Iodine-125 Iotrex and Iridium-192 seeds irradiating the tissues around the post-surgical cavity (by Proxima Therapeutices, Inc.) (King et al., Amer J Surg 180:299-304, 2000; Vicini et al., J Clin Oncol 19:1993, 2002). This approach has also recently gained FDA approval, for example GlialSite for brain tumors and MammoSite for breast cancer (deGuzman et al., J Nucl Med 41 (5 Suppl):274P, 2000; Dempsey et al., Int J Radiat Oncol Biol Phys 42:421-29, 1998). These FDA approved approaches illustrate the desirable features of locoregional radionuclide therapy: predictable dosimetry, monitoring capability, and short duration. But a more desirable approach of bypassing surgical resection and directly ablating tumors using, for example, intratumoral injection of radionuclides has not been found. This lack of alternative approaches is due to the lack of requisite information on radionuclide dispersion and on radiation dosimetry in the tumor and surrounding tissues to establish efficacy and safety.

The Medical Internal Radionuclide Dosimetry (MIRD) schemes require accurate determination of volume and residence time of dispersed radionuclides (Loevinger et al., MIRD Primer. Society of Nuclear Medicine, 1991, incorporated herein by reference). A recent report directly measured the injectate volume using the full-width half maximum (FWHM) of the injection site from the scintigram. The accuracy of this volume estimate was limited by the system resolution of 2 cm (Hung et al., J Nucl Med 356:1895-1901, 1995). The search for an accurate measurement of the dispersed injectate volume for dosimetry has been futile because, besides the radioactivity, there is no other physical signal from the injected radionuclide for external imaging. Thus, a radiopharmaceutical that can provide signals for volumetric measurements and gamma rays for radioactivity measurements is highly desirable because it can be applied to study the movement or sequestration of particles in tumors and to derive the radiation dosimetry of the radionuclides.

Thus, it is highly desirable to generate a radiopharmaceutical that can be used for locoregional treatment of abnormal tissues while simultaneously allowing for more accurate measurements of the radiation dosimetry to the treated tissue, for example a tumor, by accurately measuring the radioactivity distribution and volume distribution parameters of the radiopharmaceutical.

### BRIEF SUMMARY OF THE INVENTION

The present disclosure is directed to a radiopharmaceutical macroaggregate composition for the treatment of abnormal tissue comprising particles having a minimum size of one micron, wherein the particles comprise a metal and one or more radioactive isotopes, and have sufficient radioactivity for locoregional ablation of cells in the abnormal tissue. In other preferred embodiments, the radiopharmaceutical macroaggregate composition is used to treat abnormal cells. Preferably the metal in the particles is iron, gadolinium, or calcium. When the particles includes iron or gadolinium, the radiopharmaceutical macroaggregate composition is paramagnetic. In other preferred embodiments of the present disclosure, the one or more radioactive isotopes in the particles are selected from the group consisting of Gallium-67 (⁶⁷Ga), Yttrium-90 (⁹⁰Y), Gallium-68 (⁶⁸Ga), Thallium-201 (²⁰¹Tl), Strontium-89 (⁸⁹Sr), Indium-111 (¹¹¹In), Iodine-131 (¹³¹I), Samarium-153 (¹⁵³Sm), Technetium-99m (^{99m}Tc), Rhenium-186 (¹⁸⁶Re), Rhenium-188 (¹⁸⁸Re), Copper-62 (⁶²Cu), and Copper-64 (⁶⁴Cu). Preferably the radioactive isotope(s) in the composition emit beta radiations, gamma radiations, and/or positrons.

Another preferred embodiment of the present disclosure is a paramagnetic radiopharmaceutical macroaggregate generated by co-precipitation or the mechanism of adsorption of nonradioactive particles with radioactive isotopes, which provides magnetic signals for volumetric measurements and gamma rays for radioactivity measurements. In a preferred embodiment, a nonradioactive metal particle, for example Iron (Fe) or Gadolinium (Gd) is co-precipitated the radioactive isotopes, for example ⁶⁷Ga, ⁹⁰Y, ⁶⁸Ga, ²⁰¹Tl, ⁸⁹Sr, ¹¹¹In, ¹³¹I, ¹⁶⁶Ho, ¹⁵³Sm, ^{99m}Tc, ¹⁸⁶Re, ¹⁸⁸Re, ⁶²Cu, and ⁶⁴Cu. In another preferred embodiment, the paramagnetic radiopharmaceutical macroaggregate is generated by the mechanism of adsorption of radioactive isotopes by nonradioactive particles. These paramagnetic radiopharmaceuticals can be used to study the movement or sequestration of particles in a tumor and to derive the radiation dosimetry of the particles. The paramagnetic properties of the radiopharmaceutical macroaggregate allows for the accurate measurement of geographic distribution of the radiopharmaceutical macroaggregate in the injected and surrounding tissues. Measuring the radioactivity of the same radiopharmaceutical macroaggregate allows for the measurement of radioactivity and retention in the same tissues. These properties allow for locoregional treatment of abnormal tissues with the paramagnetic radiopharmaceutical macroaggregate while simultaneously allowing for more accurate measurements of the radiation dosimetry to the treated tissue.

In another preferred embodiment, a nonparamagnetic radiopharmaceutical macroaggregate is generated by co-precipitating nonradioactive particles, for example Calcium (Ca), with radioactive isotopes, for example ⁶⁷Ga, ⁹⁰Y, ⁶⁸Ga, ²⁰¹Tl, ⁸⁹Sr, ¹¹¹In, ¹³¹I, ¹⁶⁶Ho, ¹⁵³Sm, ¹⁸⁶Re, ¹⁸⁸Re, ^{99m}Tc, ⁶²Cu, and ⁶⁴Cu. In another preferred embodiment, the nonparamagnetic radiopharmaceutical macroaggregate is generated by the mechanism of adsorption of radioactive isotopes by nonradioactive particles. Although the Calcium radiopharmaceutical macroaggregate do not have paramagnetic properties, they are biodegradable because the calcium hydroxide particles are dissolved and reabsorbed by surrounding tissues. The Calcium radiopharmaceutical macroaggregate can also be localized using a Computed Tomography (CT) scanner. Localization of the radiopharmaceutical macroaggregate may also be monitored by ultrasonography.

In yet another preferred embodiment, the radiopharmaceutical macroaggregates includes particulates or microspheres, for example particulates or microspheres that are small hollow or cup-shaped ceramic particles or glass microspheres. In preferred embodiments the ceramic base material of the particulates or microspheres is made of alumina, zirconia, silica, or combinations thereof. In a preferred embodiment, a non-radioactive metal is co-precipitated with one or more radioactive isotopes and ceramic base material or glass to generate the particulate or microsphere radiopharmaceutical macroaggregates. In another preferred embodiment, a non-radioactive metal and one or more radioactive isotopes are adsorbed by ceramic base material or glass to generate the particulate or microsphere radiopharmaceutical macroaggregates. In a preferred embodiment, the non-radioactive metal is Ca, Fe, or Gd. In preferred embodiment, the radioactive isotope(s) used to produce the radiopharmaceutical macroaggregate include but are not limited to ⁶⁷Ga, ⁹⁰Y, ⁶⁸Ga, ²⁰¹Tl, ⁸⁹Sr, ¹¹¹In, ¹³¹I, ¹⁶⁶Ho, ¹⁵³Sm, ¹⁸⁶Re, ¹⁸⁸Re, ^{99m}Tc, ¹²³I, ¹³¹I, ⁶²Cu, and ⁶⁴Cu. In other preferred embodiments, the size of the particulate or microsphere radiopharmaceutical macroaggregates is from about 1 to about 200 microns, more preferably from about 5 to about 80 microns in size.

In preferred embodiments, the radiopharmaceutical macroaggregate disperses after injection into the abnormal tissue, for example neoplastic tissue such as a tumor, but remains contained within the abnormal tissue. In other preferred embodiments, the radiopharmaceutical macroaggregate is used for radiosynoviorthesis. Preferably the radiation absorbed doses from the radiopharmaceutical macroaggregate will be high within the abnormal tissue to ablate abnormal cells, but low in surrounding tissues and body organs. In another preferred embodiment, magnetic resonance imaging (MRI), Positron Emission Tomography (PET), Computed Tomography (CT) scanner, ultrasonography, and/or high resolution gamma scintigraphy are used to measure the spatial and temporal profiles of the radiopharmaceutical macroaggregate after injection. The presence of ferromagnetic particles (such as iron) in the radiopharmaceutical macroaggregate also provides a convenient route for ferromagnetic local hyperthermia during or after the radioactivity decay is completed.

In yet another preferred embodiment, radiopharmaceutical macroaggregates with more than one radioactive isotopes are generated by co-precipitating the radioactive isotopes with a metal, for example Ca, Fe, or Gd. In preferred embodiments, the radioactive isotopes are selected from the group consisting of ⁶⁷Ga, ⁹⁰Y, ⁶⁸Ga, ²⁰¹Tl, ⁸⁹Sr, ¹¹¹In, ¹³¹I, ¹⁶⁶Ho, ¹⁵³Sm, ¹⁸⁶Re, ¹⁸⁸Re, ^{99m}Tc, ⁶²Cu, and ⁶⁴Cu. Preferably, double-labeled radiopharmaceutical macroaggregates are generated by co-precipitating two radioactive isotopes with one non-radioactive metal. A preferred double-labeled radiopharmaceutical macroaggregates is ⁹⁰Y-Fe-⁶⁷Ga. In another preferred embodiment, the non-radioactive metal (M) is co-precipitated with a radionuclide cation (C) and a radionuclide anion (A) to generate a double-labeled radiopharmaceutical macroaggregate (A-M-C). Preferred A-M-C radiopharmaceutical macroaggregates include ⁹⁰Y-Fe-^{99m}Tc, ⁹⁰Y-Ca-^{99m}Tc, and ⁹⁰Y-Gd-^{99m}Tc. In yet another preferred embodiment, the non-radioactive metal (M) is co-precipitated with two radionuclide cations (C1 and C2) to generate C1-M-C2. The above preferred embodiments can also be generated using the mechanism of adsorption.

In another preferred embodiment, radiopharmaceutical macroaggregates are generated by co-precipitating Phytate (P) with a non-radioactive particle and one or more radioactive isotopes. Preferably, a non-radioactive metal (M) is co-precipitated with a radionuclide cation (C) and Phytate (M-C-P), or a non-radioactive metal (M) is co-precipitated with a radionuclide anion (A) and Phytate (M-A-P). In preferred embodiments, the metal is Ca, Fe, or Gd, and the radionuclide cation is ⁶⁷Ga citrate, ⁹⁰Y chloride (Cl), ²⁰¹Tl Cl, ⁸⁹Sr Cl, ⁶²Cu Cl, ⁶⁴Cu Cl, ¹⁵³Sm EDTMP, I⁵³Sm Cl, ¹⁶⁶Ho DOTMP, ¹⁶⁶Ho Cl, ¹¹¹In Cl, or ¹¹¹In DTPA, and the radionuclide anion is ^{99m}TcO₄, ¹⁸⁶Re Perrhenate, or ¹⁸⁸Re Perrhenate. In yet another preferred embodiment, radiopharmaceutical macroaggregates are generated by precipitating Phytate with a non-radioactive metal as well as a radionuclide cation and a radionuclide anion (M-A-C-P). In another preferred embodiment, radiopharmaceutical macroaggregates are generated by precipitating Phytate with a non-radioactive metal as well as two radionuclide cations (C1 and C2) to generate (C1-M-P-C2). Preferred M-A-C-P and C1-M-P-C2 radiopharmaceutical macroaggregates generated include Fe-^{99m}Tc-⁹⁰Y-P, Gd-^{99m}Tc-⁹⁰Y-P, Ca-^{99m}Tc-⁹⁰Y-P, Fe-⁶⁷Ga ⁹⁰Yp Gd-⁶⁷Ga-⁹⁰Yp, Ca-⁶⁷Ga-⁹⁰Y-P, Fe-^{99m}Tc-¹¹¹In-P, Ca-^{99m}Tc-¹¹¹In-P, Gd-^{99m}Tc-¹¹¹In-P, Fe-^{99m}Tc-67 Ga-P, Ca-^{99m}Tc-⁶⁷Ga-P, Gd-^{99m}Tc-⁶⁷Ga-P, Fe-⁹⁰Y-¹¹¹In-P, Ca-⁹⁰Y-¹¹¹In-P, and Gd-⁹⁰Y-¹¹¹In-P.

In a preferred embodiment of the present disclosure the particles in the radiopharmaceutical macroaggregate composition are composed of a metal and one radioactive isotope. Preferably the radioactive isotope is a cation or an anion. In another preferred embodiment the particles are composed of a metal and two radioactive isotopes. Preferably the two radioactive isotopes are either both cations, both anions, or one is a cation and one is an anion; more preferably one of the radioactive isotopes is Holmium-166 (¹⁶⁶Ho). In yet another preferred embodiment the particles further comprise Phytate. Preferably the metal to radioactive isotope(s) molar ratio is about 10⁶:1. In other preferred embodiments, the particles are biodegradable. The preferable size of the particles is from about 5 to about 50 microns.

Preferred embodiment of the present disclosure are methods for the locoregional treatment of abnormal tissue, comprising administering a radiopharmaceutical macroaggregate composition to a subject in the region of the abnormal tissue, wherein the radiopharmaceutical macroaggregate composition comprises particles having a minimum size of one micron, wherein the particles comprise a metal and one or more radioactive isotopes, and have an effective amount of radioactivity for locoregional ablation of cells in the abnormal tissue. Preferably the subject is a vertebrate such as a mammal, more preferably the subject is an animal, and most preferably the subject is human. In other preferred embodiments, the radiopharmaceutical macroaggregate composition is utilized for Selective Internal Radiation Therapy (SIRT). Preferably the radiopharmaceutical macroaggregate composition is administered by intra-arterial injection. In other preferred embodiments, the abnormal tissue is a neoplasm or synovial tissue, more preferably the neoplasm is a tumor. For tumors, the radiopharmaceutical macroaggregate composition is preferably administered directly into the tumor by injection. In other preferred embodiments, a macroaggregate composition containing Gd (with or without attached radionuclide(s)) is exposed to neutron irradiation for the locoregional treatment of abnormal tissue.

In other preferred embodiments, the radiopharmaceutical macroaggregate composition is administered by injection, for example intratumoral, intravenous, intravascular, intraparenchymal, intraarterial, intracavitary, intra-pleural, intraperitonal, or intrathecal injection. The radiopharmaceutical macroaggregate composition may be injected at a single location, or multiple locoregional injections may be used in different locations in the same subject, for example, there may be multiple injection sites in a single tumor. If multiple injections of the radiopharmaceutical macroaggregate composition are administered to a subject, they may be given at the same time, or over a period of time (fractionation), for effective treatment.

In other preferred embodiments of the present disclosure, the one or more radioactive isotopes in the particles are selected from the group consisting of Gallium-67 (⁶⁷Ga), Yttrium-90 (⁹⁰Y), Gallium-68 (⁶⁸Ga), Thallium-201 (²⁰¹Tl), Strontium-89 (⁸⁹Sr), Indium-111 (¹¹¹In), Iodine-131 (¹³¹I), Samarium-153 (¹⁵³Sm), Holmium-166 (¹⁶⁶Ho), Technetium-99m (^{99m}Tc), Rhenium-186 (¹⁸⁶Re), Rhenium-188 (¹⁸⁸Re), Copper-62 (⁶²Cu), and Copper-64 (⁶⁴Cu). In yet another preferred embodiment the particles further comprise Phytate. Preferably the metal in the particles is iron, gadolinium, or calcium. When the particles includes iron or gadolinium, the radiopharmaceutical macroaggregate composition is paramagnetic. The paramagnetic properties of the radiopharmaceutical macroaggregate composition preferably are used to measure the geographic distribution and derive radiation dosimetry of the radioactive composition. In radiopharmaceutical macroaggregate compositions that include iron in the particles, the ferromagnetic properties of the iron is used for local hyperthermia therapy. Preferably magnetic resonance imaging (MRI), Positron Emission Tomography (PET), ultrasonography, or high resolution gamma scintigraphy is used to measure the spatial and temporal profiles of the paramagnetic composition. A Computed Tomography (CT) scanner is preferably used to localize radiopharmaceutical macroaggregate compositions that include calcium.

A preferred embodiment of the present disclosure is a radiopharmaceutical macroaggregate composition for the treatment of abnormal tissue comprising particles having a minimum size of one micron, wherein the particles comprise a metal and one or more radioactive isotopes, and have sufficient radioactivity for locoregional ablation of cells in the abnormal, produced by co-preciptitation or the mechanism of adsorption. Preferably, the radiopharmaceutical macroaggregate composition is prepared by a process comprising the steps of:

(a) mixing one or more radioactive isotopes with a metal chloride;

(b) adding an alkaline to the mixture of part (a) to precipitate the radioactive isotopes with the metal to form the particles;

(c) separating the precipitated particles from any remaining soluble radioactive isotopes from the particles; and

(d) isolating the radioactive particles.

In preferred embodiments, the metal chloride is selected from the group consisting of ferric chloride (FeCl₃), calcium chloride (CaCl₂), and gandolinium chloride (GdCl₃). In other preferred embodiments, the alkaline is sodium hydroxide or ammonium hydroxide. Preferably, the precipitated particles are separated from any remaining soluble radioactive isotopes by centrifugation.

Another preferred embodiment of the present disclosure is a radiopharmaceutical macroaggregate composition for the treatment of abnormal tissue comprising particles having a minimum size of one micron, wherein the particles comprise a metal and one or more radioactive isotopes, and have sufficient radioactivity for locoregional ablation of cells in the abnormal, produced by a process comprising the steps of:

(a) adding an alkaline to a metal chloride to form a precipitate;

(b) mixing one or more radioactive isotopes with the precipitate of part (a) to allow the radioactive isotopes to adsorb to the precipitate and generate a radioactive precipitate;

(c) separating the radioactive precipitate of part (b) from any remaining soluble radioactive isotopes; and

(d) isolating the radioactive precipitate.

In preferred embodiments, the metal chloride is selected from the group consisting of ferric chloride (FeCl₃), calcium chloride (CaCl₂), and gandolinium chloride (GdCl₃). In other preferred embodiments, the alkaline is sodium hydroxide or ammonium hydroxide. Preferably, the radioactive precipitate is separated from any remaining soluble radioactive isotopes by centrifugation.

Other embodiments of the present disclosure are directed to methods of acupuncture therapy for an acupuncture-responsive condition, comprising administering a radiopharmaceutical macroaggregate composition into one or more acupuncture points of a subject, wherein the radiopharmaceutical macroaggregate composition comprises particles having a minimum size of one micron, wherein the particles comprise a metal and one or more radioactive isotopes, and have an effective amount of radioactivity to enhance the acupuncture therapy. Preferably, the subject of the acupuncture therapy is human, and the acupuncture-responsive condition is pain or rheumatoid arthritis, and the radiopharmaceutical macroaggregate composition is administered by injection into the acupuncture points.

### BRIEF DESCRIPTION OF THE SEVERAL VIEWS OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Diagram of the normalized S-values inside the 5 spheres of volumes of 0.4 cc, 2 cc, 10 cc, 50 cc, and 250 cc from Monte Carlo Simulation of gamma and beta emissions.

FIG. 2. Diagram of the 10% isodose ranges (i.e., the distance from the sphere where only 10% of the radiation dose from the sphere remains) from simulated depth dosimetry for 5 spheres of volumes of 0.4 cc, 2 cc, 10 cc, 50 cc, and 250 cc.

FIG. 3. An MRI study of the Gallium-Iron radiopharmaceutical macroaggregate (GIMA) demonstrated decreases in Gradient Echo (GRE) signals as Fe contents increased to the concentration range intended for intratumoral injection (FIGS. 3A and 3B). FIG. 3A shows a GE Signa 1.5 T MRI scanner that demonstrated decreasing GRE signals from 6 phantoms of 1 cc cylinders. FIG. 3B shows decreasing GRE signals with iron content with GRE pulse sequences but not with Fast Spin Echo (FSE) sequences.

FIG. 4. 0.1 mCi ⁶⁷Ga GIMA was injected intratumorally (IT) and intramuscularly (IM) into the left leg of a 160 gram rat with a breast tumor implanted it in its right leg. FIG. 4 illustrates the prolonged retention of ⁶⁷Ga GIMA (65-80% at 18 hours) at both the intramuscular and intratumoral injection sites. A ⁶⁷Ga standard was placed in the upper left corner of FIG. 4 as a positive control. Persistently low (<2%) lung uptake was also present in the rat.

FIG. 5. Graph of the in vivo rat tumor growth rates after treatment with the radiopharmaceutical macroaggregate GIMA. On day zero, 100,000 rat mammary cancer 13762F tumor cells were implanted into the right thigh muscle of Fischer 344 female rats. In one set of experiments, on day 10 the rats injected with tumor cells were subsequently treated with 0.2 mCi or 0.8 mCi of GIMA after the tumors became palpable (0.2 mCi IT Day 10 and 0.8 mCi IT Day 10 respectively). In another set of experiments, 1 mCi of GIMA was injected intramuscularly on day 3 into the same location on the right thigh of the rats that the tumor cells had been injected into (1 mCi IT Day 3). The remaining rats injected with tumor cells were used as controls (Control2). Tumor sizes were monitored regularly and the in vivo tumor growth rates over time are shown.

### DETAILED DESCRIPTION OF THE INVENTION

This present disclosure is directed to radiopharmaceuticals that are generated by co-precipitating nonradioactive particles with radioactive isotopes to produce a radiopharmaceutical macroaggregate. In another embodiment, the radiopharmaceuticals of the present disclosure are generated by the mechanism of adsorption of radioactive isotopes by nonradioactive particles to produce a radiopharmaceutical macroaggregate. As used herein, the term “radiopharmaceutical macroaggregate(s)” includes both paramagnetic radiopharmaceuticals and nonparamagnetic radiopharmaceuticals. These radiopharmaceutical macroaggregates are used for locoregional ablation of abnormal tissue, preferably neoplastic tissue, cancerous tissue, tumors, or synovial tissues. A significant advantage of these radiopharmaceutical macroaggregates for therapeutic applications is that the co-precipitated nonradioactive particles allow for measurements of the distribution and dosimetry of the radiopharmaceutical macroaggregates after they have been introduced, preferably by injection, into a subject. As used herein, the term “subject” refers to mammals, preferably humans. As used herein, “radioactive isotope(s)” are also referred to as “radionuclide(s).” In a preferred embodiment a single radioactive isotope is used to produce a radiopharmaceutical macroaggregate. In another preferred embodiment, two or more radioactive isotopes are used to produce a radiopharmaceutical macroaggregate.

In the present disclosure, paramagnetic radiopharmaceutical macroaggregates are generated by co-precipitating nonradioactive particles with radioactive isotopes to produce a paramagnetic radiopharmaceutical macroaggregate, which provides magnetic signals for volumetric measurements of geographic distribution of the macroaggregate in injected and surrounding tissues, and gamma rays for radioactivity measurements in the same tissues. Alternatively, paramagnetic radiopharmaceutical macroaggregates are generated by the mechanism of adsorption of radioactive isotopes by nonradioactive particles. In preferred embodiments, paramagnetic radiopharmaceutical macroaggregates are generated using nonradioactive particles such as metals, including but not limited to Iron (Fe) or Gadolinium (Gd). The radioactive isotopes that can be co-precipitated with or adsorbed by nonradioactive particles to produce a paramagnetic radiopharmaceutical macroaggregate include but are not limited to Gallium-67 (⁶⁷Ga), Yttrium-90 (⁹⁰Y), Gallium-68 (⁶⁸Ga), Thallium-201 (²⁰¹Tl), Strontium-89 (⁸⁹Sr), Indium-111 (¹¹¹In), Iodine-131 (¹³¹I), Holmium-166 (¹⁶⁶Ho), Samarium-153 (¹⁵³Sm), Rhenium-186 (¹⁸⁶Re), Rhenium-188 (⁸⁸Re), Technetium-99m (^{99m}Tc), Copper-62 (⁶²Cu), and Copper-64 (⁶⁴Cu). Preferably, the radioactive isotopes are either the cationic and anionic species of the radionuclide.

Important properties of the radioactive isotopes that may be used to generate radiopharmaceutical macroaggregates of the present disclosure are set forth below in Table 1:

TABLE 1

| | | Principal Gammas | Principal Betas |
|---|---|---|---|
| Radionuclide | Half Life | (MeV) | (MeV) |
| Cu62 | 9.74 minutes | 0.511 | |
| Cu64 | 12.7 hours | 0.511 | 0.578 |
| Ga67 | 78 hours | 0.093 | |
| Ga68 | 68 minutes | 0.511 | |
| Ho166 | 26.8 hours | 0.184 | 0.072 |
| I123 | 13.2 hours | 0.159 | |
| I131 | 8.04 hours | 0.364 | 0.606 |
| In111 | 68 hours | 0.245 | |
| Re186 | 3.72 days | 0.14 | 0.323 |
| Re188 | 16.8 hours | 0.16 | 0.8 |
| Sm153 | 46.3 hours | 0.042 | 0.702 |
| Sr89 | 50.55 days | 0.909 | 1.491 |
| Tc99m | 6.02 hours | 0.141 | |
| TI201 | 73.1 hours | 0.071 | |
| Y86 | 14.4 hours | | 1.4 |
| Y90 | 64 hours | | 2.284 |

Preferably the paramagnetic radiopharmaceutical macroaggregate emits beta and/or alpha radiation sufficient to ablate abnormal cells, and may or may not emit gamma rays. In preferred embodiments, the radiopharmaceutical macroaggregate yield about 80-99% radioactivity that is stable in phosphate buffer saline over at least 24 hours. The paramagnetic nature and/or metal densities of the precipitates allows for the localization and quantification of the particles in vivo as well as accurate dosimetric estimates, while the radioactive nature of the particles provides signals for localization and measurement of radioactivities, as well as locoregional ablation of abnormal tissues.

In other preferred embodiment, magnetic resonance imaging (MRI), Positron Emission Tomography (PET), ultrasonography, and/or high resolution gamma scintigraphy are used to measure the spatial and temporal profiles of the paramagnetic radiopharmaceutical macroaggregate after injection, and to determine the effective half-life, biological half-life, and residence time of the paramagnetic radiopharmaceutical macroaggregate. For example, recent advancements in magnetic and nuclear imaging technologies have enabled measurements of small volumes of iron in small quantities in the body non-invasively (Bonkovsky et al., Radiology 212(1):227-234, 1999, incorporated herein by reference). Preferably, the pharmacokinetic data generated using such techniques combined with nuclear imaging is used to calculate whole-body, organ, and locoregional radiation dosimetry to evaluate the safety and efficacy factors for a specific paramagnetic radiopharmaceutical macroaggregate.

The paramagnetic properties of Iron or Gadolinium in paramagnetic radiopharmaceutical macroaggregates allow for the localization and quantification of the macroaggregates using an MRI scanner, both in vitro and in vivo. MRI is an important diagnostic tool that exploits the differences in relaxation rates of water protons in different tissues, translating these differences into three-dimensional anatomic information. Paramagnetic metal complexes can shorten proton relaxation times and provide improved tissue contrast depending on their biodistribution when administered in vivo (Koenig, Isr J Chem 28:345, 1988). The supramagnetic properties can also be used for the mobilization of the macroaggregates through externally applied magnetic fields (Alexiou et al., Cancer Research 60(23):6641-48, 2000; Rudge et al., Biomaterials 21(14):1411-20, 2000; incorporated herein by reference). Alternatively, the high concentration of metal in the precipitate can be measured using a Computed Tomography (CT) scanner.

Furthermore, the presence of ferromagnetic iron in the radiopharmaceutical macroaggregates also provides a convenient route for local hyperthermia during or after the radioactivity decay is completed (Steeves et al., Int J Hyperthermia 8:443-49, 1992; Suzuki et al., Nippon Gan Chiryo Gakkai Shi 25(11):2649-58, 1990; Moroz et al., Int J Hyperthermia 18:129-40, 2002; Eikesdal et al., Int J Hyperthermia 18:141-52, 2002; Jones et al., Int J Hyperthermia 18:117-128; Granov et al., An angiographic ferromagnetic embolization and a local high-frequency hyperthermia in the therapy of renal cell carcinoma. Methodical recommendations. St. Petersburg University Press. 1-10, 2000; incorporated herein by reference). For example, the ferromagnetic properties of iron co-precipitates allows for concurrent or subsequent local hyperthermia when the injected subject is exposed to an alternating magnetic field, thereby achieving maximum therapeutic effects while avoiding toxicity (Li et al., J Nucl Med 43(5):370P, 2002, incorporated herein by reference). This local hyperthermia therapy can be applied either concurrently or subsequently to the introduction of the ferromagnetic particles into the patient to increase the effectiveness of the neoplastic, cancer or tumor therapy.

Alternatively, nonparamagnetic radiopharmaceuticals are generated using Calcium to co-precipitate or adsorb the radioactive isotopes. These nonparamagnetic radiopharmaceuticals do not have paramagnetic properties, but may be biodegradable through the resorption of calcium hydroxide particles by surrounding tissues. Preferably the nonparamagnetic radiopharmaceuticals are reabsorbed after the radioactive decay of the radioisotope is completed. These calcium containing particles can be localized using a CT scanner. The radioactive isotopes that can be used to generate these nonparamagnetic radiopharmaceutical macroaggregate include but are not limited to ⁶⁷Ga, ⁹⁰Y ⁶⁸Ga, ²⁰¹T, ⁸⁹Sr, ¹¹¹In, ¹³¹I, ¹⁶⁶Ho, ¹⁵³Sm, ¹⁸⁶Re, ¹⁸⁸Re, ^{99m}Tc, ¹²³I, ¹³¹I, ⁶²Cu, and ⁶⁴Cu. The radioactive isotopes can include either or both of the cationic and anionic species of the radionuclide.

Radiopharmaceutical macroaggregates can also be generated by co-precipitating or adsorbing more than one radionuclide with a metal, for example double-labeled radiopharmaceutical macroaggregates generated by co-precipitation or adsorption of two radionuclide isotopes with one non-radioactive metal. In a preferred embodiment, the non-radioactive metal is Ca, Fe, or Gd. In another preferred embodiments, the radionuclides are selected from the group consisting of ⁶⁷Ga, ⁹⁰Y, ⁶⁸Ga, ²⁰¹Tl, ⁸⁹Sr, ¹¹¹In, ¹³¹I, ¹⁶⁶Ho, ¹⁵³Sm, ¹⁸⁶Re, ¹⁸⁸Re, ^{99m}Tc, ¹²³I, ¹³¹I, ⁶²Cu, and ⁶⁴Cu. Preferred double-labeled radiopharmaceutical macroaggregates include but are not limited to ⁹⁰Y-Fe-⁶⁷Ga, ⁹⁰Y-Ca-⁶⁷Ga, ⁹⁰Y-Gd-⁶⁷Ga, ⁹⁰Y-Fe-¹¹¹In, ⁹⁰Y-Ca-¹¹¹In, and ⁹⁰Y-Gd-¹¹¹In. In another preferred embodiment, the non-radioactive metal (M) is co-precipitated with a radionuclide cation (C) and a radionuclide anion (A) to generate a double-labeled radiopharmaceutical macroaggregate (A-M-C). Preferred A-M-C radiopharmaceutical macroaggregates include ⁹⁰Y-Fe-^{99m}Tc, ⁹⁰Y-Ca-^{99m}Tc, and ⁹⁰Y-Gd-^{99m}Tc. In yet another preferred embodiment, two radionuclide cations (C1 and C2) are precipitated with non-radioactive M (C1-M-C2). In another preferred embodiment, two radionuclide anions (A1 and A2) are precipitated with non-radioactive M (A1-M-A2). The above preferred embodiments can also be generated using the mechanism of adsorption.

Another method for producing radiopharmaceutical macroaggregates involves the use of Phytate (C₆H₁₂O₁₈P₆, or P). Phytate is also known as Inositol hexaphosphate (IP-6) and phytic acid. In a preferred embodiment, a non-radioactive metal (M) is co-precipitated with a radionuclide cation (C) and Phytate (M-C-P). Preferably the metal is Ca, Fe, or Gd, and the radionuclide cation is ⁶⁷Ga citrate, ⁹⁰Y chloride (Cl), ⁶⁸Ga citrate, ²⁰¹Tl Cl, ⁸⁹Sr Cl, ⁶²Cu Cl, ⁶⁴Cu Cl, ¹⁵³Sm EDTMP, ¹⁵³Sm Cl, ¹⁶⁶Ho DOTMP, ¹⁶⁶Ho Cl, ¹¹¹In Cl, or ¹¹¹In DTPA. In another preferred embodiment, a metal and a radionuclide anion (A) are co-precipitated with Phytate (M-A-P). Preferably the radionuclide anion is ^{99m}TcO₄, ¹⁸⁶Re Perrhenate, or ¹⁸⁸Re Perrhenate. In yet another preferred embodiment, radiopharmaceutical macroaggregates are generated by precipitating Phytate with a metal as well as a radionuclide cation and a radionuclide anion (M-A-C-P). In yet another preferred embodiment, radiopharmaceutical macroaggregates are generated by precipitating Phytate with a non-radioactive metal as well as two radionuclide cations (C1 and C2) to generate (C1-M-P-C2). In another preferred embodiment, radiopharmaceutical macroaggregates are generated by precipitating Phytate with a non-radioactive metal as well as two radionuclide anions (A1 and A2) to generate (A1-M-P-A2). The above preferred embodiments can also be generated using the mechanism of adsorption.

Preferred M-A-C-P and C1-M-P-C2 radiopharmaceutical macroaggregates generated include Fe-^{99m}Tc-⁹⁰Y-P, Gd-^{99m}Tc-⁹⁰Y-P, Ca-^{99m}Tc-⁹⁰Y-P, Fe-⁶⁷Ga-⁹⁰Y-P, Gd-⁶⁷Ga-90-P, Ca-⁶⁷Ga-⁹⁰Y-P, Fe-⁹⁰Y-¹¹¹In-P, Ca-⁹⁰Y-¹¹¹In-P, Gd-⁹⁰Y-¹¹¹In-P, Fe-^{99m}Tc-¹¹¹In-P, Ca-^{99m}Tc-¹¹¹In-P, Gd-^{99m}Tc-¹¹¹In-P, Fe-^{99m}Tc-⁶⁷Ga-P, Ca-^{99m}Tc-⁶⁷Ga-P, and Gd-^{99m}Tc-⁶⁷Ga-P. The paramagnetic and ferromagnetic properties of the non-radioactivity moiety in these radiopharmaceutical macroaggregates are conserved in the M-C-P, M-A-P, C1-M-P-C2, and M-A-C-P co-precipitates. For M-A-C-P radiopharmaceutical macroaggregates, it appears that one radionuclide (C or A) is linked to another radionuclide (A or C) through the relatively inert P, as well as an M.

The A-M-C, C1-M-C2, M-C-P, M-A-P, C1-M-P-C2, and M-A-C-P radiopharmaceutical macroaggregates offer many potential therapeutic advantages. For example, ⁹⁰Y, which emits beta rays, is well suited for radiotherapy, but has poor imaging characteristics for monitoring. Therefore, another radionuclide, for example ^{99m}Tc, ¹¹¹In, or ⁶⁷Ga, which emits gamma rays and is well suited for monitoring, can be co-precipitated with ⁹⁰Y to generate a radiopharmaceutical macroaggregates with both desirable characteristics. Additionally, different therapeutic radionuclides with various half-lives and ranges can be co-precipitated to provide various spectrum for ablating abnormal tissue.

Another method for producing radiopharmaceutical macroaggregates involves particulates or microspheres, for example particulates or microspheres that are small hollow or cup-shaped ceramic particles or glass microspheres (U.S. Pat. Nos. 6,537,518, 6258,338, and 4,789,501, incorporated herein by reference). In preferred embodiments the ceramic base material of the particulates or microspheres is made of alumina, zirconia, silica, or combinations thereof. In a preferred embodiment, a non-radioactive metal is co-precipitated with a radioactive isotope and ceramic base material or glass to generate the particulate or microsphere radiopharmaceutical macroaggregates. In another preferred embodiment, a non-radioactive metal and a radioactive isotope are adsorbed by ceramic base material or glass to generate the particulate or microsphere radiopharmaceutical macroaggregates. In a preferred embodiment, the non-radioactive metal is Ca, Fe, or Gd. Preferably one or more radioactive isotopes are used to generate the radiopharmaceutical macroaggregate. In preferred embodiment, the radioactive isotope(s) used to produce the radiopharmaceutical macroaggregate include but are not limited to ⁶⁷Ga, ⁹⁰Y, ⁶⁸Ga, ²⁰¹Tl, ⁸⁹Sr, ¹¹¹In, ¹³¹I, ¹⁶⁶Ho, ¹⁵³Sm, ¹⁸⁶Re, ¹⁸⁸Re, ^{99m}Tc, ¹²³I, ¹³¹I, ⁶²Cu, and ⁶⁴Cu.

In another preferred embodiment, the particulate or microsphere radiopharmaceutical macroaggregate is generated by co-precipitating a non-radioactive metal with ceramic base material or glass, as well as a base component that may be rendered radioactive by exposure to a neutron beam. For example, yttria or another yttrium-containing compound or salt of yttrium is co-precipitated to form the macroaggregate, and the macroaggregate is then exposed to a neutron beam to generate a particulate or microsphere radiopharmaceutical macroaggregate containing ⁹⁰Y. Alternatively, a non-radioactive metal and a base component may be adsorbed by ceramic base material or glass and exposed to a neutron beam to generate a radiopharmaceutical macroaggregate. In other preferred embodiments, the particulates or microspheres are made of glass, with the non-radioactive metal and the radioactive isotope(s) distributed throughout the glass.

A radiosensitizer is a drug that enhances the effect of radiation treatment in a subject. The use of a radiosensitizer (including Texaphyrin, Rhodamine, BUDR and others), whether nonradioactive or radioactive, along with radiotherapy has been found to increase tumor cell killings several fold (e.g., Teicher et al., Int J Radiat Oncol Biol Phys 13:1217-24, 1987, incorporated herein by reference). However, the systemic use of radiosensitizers is limited by low regional delivery and systemic toxicities such as hepatic and dermatologic toxicity. On the other hand, locoregional application of radiosensitizers along with locoregional radionuclide therapy with the disclosed radiopharmaceutical macroaggregates will exploit pharmacokinetic advantage because of the initial 100% exposure of the tumors to the radiosensitizer. Therefore, local injection of a radiosensitizer up to the systemic dose will have advantage of multi-fold increased delivery. Local injection of a radiosensitizer can be done before, during, or after the locoregional application of a radiopharmaceutical macroaggregate to achieve enhanced cell kills.

For example, radiopharmaceutical macroaggregates may be administered in combination with Rhodamine-123 (Rh-123). Rh-123 is a cationic, lipophilic, water-soluble oxonium chloride salt with a high affinity for the mitochondria of malignant cells. Rh-123 has been found to be selectively toxic to a number of human cancer cell lines. In a preferred embodiment, a powder form of Rh-123 is used, and as a colloid suspension, the Rh-123 will function after local injection as a slow-releasing deposit radiosensitizer, that coincides with the radioactive life of the radiopharmaceutical macroaggregate. In another preferred embodiment, a saturated solution of Rh-123 is used for locoregional injection. In a preferred embodiment, the Rh-123 is administered before, with, or after a radiopharmaceutical macroaggregate. In one preferred embodiment, a non-radioactive metal (M), a radionuclide anion and/or radionuclide cation, and Rh-123 are co-precipitated to generate a radiopharmaceutical macroaggregate. In another preferred embodiment, a radiopharmaceutical macroaggregate is generated by precipitating Rh-123 with a metal as well as one or more radionuclide cations and/or radionuclide anions. In another preferred embodiment, a non-radioactive metal (M), one or more radionuclide anions and/or one or more radionuclide cations, and Rh-123 form a radiopharmaceutical macroaggregate through the mechanism of adsorption. Preferably the metal is Ca, Fe, or Gd.

Generally, co-precipitated radiopharmaceutical macroaggregates are generated by mixing 10-100 μCi of a radioactive isotope with the metal, for example a metal chloride (FeCl₃, CaCl₂, GdCl₃), with an alkaline, for example sodium hydroxide or ammonium hydroxide (NaOH or NH₄OH, respectively). Preferably the NaOH or NH₄OH are added to reach a final pH of about 7.0 to 9.0. Alternatively the final pH can be in the range from about 3.0 to 11.0. The reactions typically occur at room temperature, although the reaction can occur at a broad range of temperatures, for example 0° C., 10° C., 20° C., 30° C., 40° C., 50° C., 60° C., 70° C., 80° C., 90° C., or 100° C. Finally, a buffer, for example Phosphate Buffered Saline (PBS) or saline, may be added to the reaction. Preferably the PBS has a pH of about 7.0, 7.4, or 8.0. The co-precipitated radiopharmaceutical macroaggregates are then separated from remaining soluble radionuclides by centrifugation or filtration.

Surprisingly, the co-precipitation of nonradioactive particles with radioactive isotopes concentrates the radioisotopes up to 100 fold in the radiopharmaceutical macroaggregates generated. This concentration of the radioisotopes allows for the production of therapeutic radiopharmaceuticals for locoregional treatment in sufficiently small volumes for practical use. The radioactive isotopes used preferably have no non-carrier added, which means that the radioactive isotopes are not mixed with like non-radioactive stable isotopes. Preferably the specific activity of the radioactive isotopes is very high (e.g., 1000 Ci/mmole). The radioactive isotopes may be diluted to some degree as long as the specific activity of the isotope is still high. In preferred embodiments, the metal to radionuclides molar ratio in the radiopharmaceutical macroaggregates is about <106:1. In other preferred embodiments the metal to radionuclides molar ratios are about <103:1, 104:1, 105:1, 107:1, 108:1, or 109:1.

Generally, radiopharmaceutical macroaggregates generated by the mechanism of adsorption are prepared by first generating stock solutions of a metal (e.g., to a final concentration 1 mg/ml), for example a metal chloride (FeCl₃, CaCl₂, GdCl₃). The metal stock solutions are then titrated with an alkaline, for example NaOH or NH₄OH, to a pH of about 7.0 to 9.0, preferably 8.0, to form a precipitate. Alternatively the final pH can be in the range from about 6.0 to 13.0. The reactions typically occur at room temperature, although the reaction can occur at a broad range of temperatures, for example 0° C., 10° C., 20° C., 30° C., 40° C., 50° C., 60° C., 70° C., 80° C., 90° C., or 100° C. Next, a buffer, for example PBS or saline, is added to the reaction, and the precipitate is centrifuged. Preferably the PBS has a pH of about 7.0, 7.4, or 8.0. Next, a small volume of radioactive isotope (preferably 1-100 μCi, more preferably 1-25 μCi, most preferably 1-2 μCi) is added to the precipitate, the reaction is washed with a buffer such as PBS, and any remaining soluble radionuclides are separated from the radioactive precipitate by centrifugation or filtration. For example, ferric hydroxide precipitates are formed using the above protocol (Pal: Granular Ferric Hydroxide for Elimination of Arsenic from Drinking Water, http://www.unu.edu/env/Arsenic/Pal.pdf, incorporated herein by reference), and one or more radionuclides are added to the precipitates to form a paramagnetic radiopharmaceutical macroaggregate. In other preferred embodiments, reducing agents (e.g., SnCl₂) or oxidizing agents (e.g., H₂O₂ or iodogen) can be used to produce higher reactivity for generating radiopharmaceutical macroaggregates. For therapeutic purposes, larger amounts (1-100 mCi) of radioactivity can be prepared in a similar fashion.

Preferably the radiopharmaceutical macroaggregate emits beta and/or alpha radiation sufficient to ablate abnormal cells, and may or may not emit gamma rays. In other preferred embodiments, the radiopharmaceutical macroaggregate emits radiation of high energy and short range, for example photons, beta particles, or other therapeutic rays. In preferred embodiments, the radiopharmaceutical macroaggregate yield about 80-99% radioactivity that is stable in phosphate buffer saline over at least 24 hours. In other preferred embodiments, the radiopharmaceutical macroaggregate yield about 81%, 82%, 83%, 84%, 85%, 86%, 87%, 88%, 89%, 90%, 91%, 92%, 93%, 94%, 95%, 96%, 97%, or 98% radioactivity that is stable in phosphate buffer saline over at least 24 hours.

In other preferred embodiments, the generated radiopharmaceutical macroaggregates have radioactivity levels of about 1 microcurie (μCi) to about 500 mCi, more preferably radioactivity levels of about 5, 10, 20, 30, 40, 50, 60, 70, 80, 90, 100, 150, 200, 250, 300, 350 400, 450, or 500 μCi to about 1, 2, 3, 4, 5, 10, 15, 20, 25, 30, 35, 40, 45, 50, 55, 60, 65, 70, 75, 80, 85, 90, 95, 100, 150, 200, 250, 300, 350, 400, or 450 mCi. A curie (Ci) is the basic unit used to describe the intensity of radioactivity in a sample of material. The curie is equal to 37 billion (3.7×10¹³ disintegrations per second, which is approximately the activity of 1 gram of radium. A curie is also a quantity of any radionuclide that decays at a rate of 37 billion disintegrations per second. In preferred embodiments, the radiation absorbed by a subject from a radiopharmaceutical macroaggregate generated according to the present disclosure is from about 1 to 500 Gray (Gy), more preferably about 2, 3, 4, 5, 10, 15, 20, 25, 30, 35, 40, 45, 50, 55, 60, 65, 70, 75, 80, 85, 90, 95, 100, 125, 150, 175, 200, 250, 300, 350, 400, or 450 Gy. In other preferred embodiments, dose penetration will be determined by the 10% isodose range (distance from the edge of the lesion where the radiation absorbed dose is 10% that inside the lesion). Preferably the range will be, for example, for the targeted abnormal tissue (e.g., lesion) itself and preferably about a 0.5 to 2 cm margin beyond the targeted abnormal tissue, more preferably about a 1 to 1.5 cm margin beyond the targeted abnormal tissue.

Preferably the co-precipitated particles produce large colloids of about 1-100 microns, more preferably about 5, 10, 15, 20, 25, 30, 35, 40, 45, 50, 55, 60, 65, 70, 75, 80, 85, 90, or 95 microns. Thus, the physical form of the radiopharmaceutical macroaggregates is preferably an amorphous colloid solution that is very flexible when injected into different locations to cover the treatment area in a subject. In another preferred embodiment, the radiopharmaceutical macroaggregates are preferably particulates or microspheres of about 1-250 microns, more preferably about 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, or 250 microns.

Preferably the radiopharmaceutical macroaggregates are unsealed radionuclides without physical containment. These radiopharmaceutical macroaggregates can be used for tumor ablation by locoregional injection, for example, by intratumoral injection, intravenous injection, intravascular injection, intraparenchymal injection, intraarterial injection, intracavitary injection, intra-pleural injection, intraperitonal injection, or intrathecal injection. For example, targeted ablation of abnormal tissues is achieved when these pharmaceuticals are delivered intravascularly or intraparenchymally because the size of the particles are large enough (>1 micrometer) to preclude being dislodged from the capillary bed or escaping through the lymphatic system. In other preferred embodiments, the radiopharmaceutical macroaggregates are applied topically to the skin, subcutaneously, or intradermally. The term locoregional primarily refers to sequestration of radionuclides from all of these routes of administration. After the radioactivity of the radiopharmaceutical macroaggregate decays, the significant residuals are only hydroxides of the nonradioactive particles used, for example Fe, Gd, or Ca, which are relatively inert or slowly biodegradable. The radiopharmaceutical macroaggregate composition may be administered by any of the above routes at a single location, or in several different locations in the same subject, for example, there may be multiple injection sites in a single tumor. If the radiopharmaceutical macroaggregate composition is administered to a subject in multiple locations, these administrations may occur at the same time, or over a period of time (fractionation), for effective treatment.

In another preferred embodiment, the radiopharmaceutical macroaggregates are administered to acupuncture points. For example, acupuncture therapy for an acupuncture-responsive condition may be achieved by administering a radiopharmaceutical macroaggregate composition into one or more acupuncture points of a subject, such that the radiopharmaceutical macroaggregate composition has an effective amount of radioactivity to enhance the acupuncture therapy. Preferably, the subject of the acupuncture therapy is human, and the radiopharmaceutical macroaggregate composition is administered by injection into the acupuncture points. Acupuncture points are well known to those of skill in the art, as set forth for example by Denmei Shudo, “Finding Effective Acupuncture Points,” Eastland Press, 2003, incorporated herein by reference. In other embodiments, the acupuncture-responsive condition is pain, rheumatoid arthritis, smoking, habit control, drug abuse control, or other acupuncture-responsive conditions well known to those of skill in the art.

In other preferred embodiments, the use of radiopharmaceutical macroaggregates to therapeutically treat a subject can be combined with other therapeutic alternatives well known to those of skill in the art for treating neoplasms, for example chemotherapy, surgery, external radiotherapy, pharmacotherapy, hormone therapy, gene therapy, radioimmunotherapy, immunotherapy, and the like (R. C. Bast, Ed. Cancer Medicine. 5th Ed. American Cancer Society, B. C. Decker, 2000, incorporated herein by reference).

Selective Internal Radiation Therapy (SIRT) involves the administration of radioactive materials, for example radioactive particulates or microspheres, into the blood supply of a target organ. SIRT has primarily been used to treat cancers of the liver. In the present disclosure, SIRT allows the radiation from the disclosed radiopharmaceutical macroaggregates to be delivered preferentially to the neoplasm in the target organ, and the radiation can be continually delivered as the radiation of earlier delivered radiopharmaceutical macroaggregates decays. The arterial blood supply can also be manipulated, for example by vasoactive substances, to direct the radiopharmaceutical macroaggregates to the cancerous part of the organ, rather than the healthy tissue of the organ (Burton et al., Europ J Cancer Clin Oncol 24:1373-76, 1988, incorporated herein by reference). Similar schemes with or without radiosensitizers may be applied with the radiopharmaceutical macroaggregates that either include or do not include Phytate.

Boron neutron-capture therapy (BNCT) of cancer is a branch of experimental radiation therapy using boron compounds containing stable isotope Boron-10 (¹⁰B). ¹⁰B is an abundant isotope (20%) with a large cross-section area (3,984 barns) to capture neutrons, which allows it to emit alpha emission for local cancer treatment (Gahbauer et al., “BNCT: A promising area of research?” Proceedings of the 5th International Conference on Applications of Nuclear Techniques: “Neutrons in Research and Industry,” Crete, Greece (1996), SPIE Proceedings Series Vol. 2867:12-22 (1997), incorporated herein by reference). The alpha-emission produced by BNCT will cause severe damage to cells in the micrometer range. For example, if the ¹⁰B is in the nucleus of a cell, BNCT will kill that cell (e.g., tumor cell) with just one-hit.

Naturally occurring Gadolinium (Gd), like Boron, has multiple stable (non-radioactive) isotopes, including Gadolinium-155 (¹⁵⁵Gd, 14.8% abundance and 68,800 barns) and Gadolinium-157 (¹⁵⁷Gd, 15.7% abundance and 250,000 barns). ¹⁵⁵Gd and ¹⁵⁷Gd are able to capture thermal neutrons and emit gamma radiations (Hofmann et al., Invest. Radiol. 34:126-33, 1999, incorporated herein by reference), thus allowing them to be used for Gadolinium neutron capture therapy (GdNCT) in cancer therapy (De Stasio et al., Cancer Res. 61:4272-4277, 2001, incorporated herein by reference). GdNCT involves systemic injection of Gd soluble compounds and neutron irradiation of the cancer region when there is peak tissue concentration of Gd. Preferably, the radiopharmaceutical macroaggregate composition utilized for GdNCT will comprise Gd chloride (GdCl₃), which will result in prolonged retention of the composition in the subject. Yoneda et al., Fundamental & Applied Toxicology, 28(1):65-70 (1995), investigated the metabolic behavior, clearance, and pulmonary effects of GdCl₃ after single intratracheal instillation in rats. Yoneda et al. observed that the Gd was deposited in the lung tissue in nonsoluble forms with an extremely long half-life. Thus, radiopharmaceutical macroaggregate composition comprising GdCl₃ will result in prolonged retention of the composition in the subject, for example, after interstitial (trachea) injection.

The current practice of BNCT and GdNCT are both limited by the delivery of the compound to the tissue by general circulation which has a low efficiency (e.g., only about 1-2% of the administered compound reaches the tumor/cancer). This limitation is overcome by the locoregional administration or application of Gd compounds to treat cancer, including but not limited to locoregional injection of Gd compounds. GdNCT compounds have a large cross-section (effectiveness) in capturing neutrons, and gamma radiation emitted from the compounds are able to kill a target cell (e.g., tumor cell) without having to enter the nucleus of the cell or even the cell itself. Therefore, locoregional administration or application of Gd compounds (e.g., intratumoral injection) in the vicinity of the targeted cells will efficiently kill the cells upon capturing of neutrons by the Gd compounds. Gd compounds include, but are not limited to, the radiopharmaceutical macroaggregates disclosed herein that include Gd. The use of these radiopharmaceutical macroaggregates or the use of nonradioactive macroaggregates or microspheres containing Gd also allow the further advantage of locating and determining the amount of Gd in a location for neutron irradiation by using gamma cameras or MRI.

In accordance with the present disclosure, “an effective amount” of the radiopharmaceutical macroaggregates is defined as an amount sufficient to ablate abnormal cells. For paramagnetic radiopharmaceutical macroaggregates, an effective amount also preferably provides magnetic signals sufficient for volumetric measurements in vivo. An effective amount of the radiopharmaceutical macroaggregates of the present disclosure may be administered in one or more injection. Effective amounts of a radiopharmaceutical macroaggregate will vary according to factors such as the degree of susceptibility of the subject, the age, sex, and weight of the subject, idiosyncratic responses of the subject, and the dosimetry of the radiopharmaceutical macroaggregate, including the level of radioactivity of the precipitated radioisotope. Optimization of such factors is well within the level of skill in the art.

There are three components that help one of skill in the art to calculate an estimation of absorbed doses to tissues surrounding the site of injection of the radiopharmaceutical macroaggregate: 1) The energy deposited in the surrounding tissues is determined using radiation transport analysis (MCNP manual, Monte Carlo N-Particle Transport Code System. RSIC 1994, incorporated herein by reference); 2) the geometry of the activity distribution (source region) is determined using MR image data; and 3) the total number of radioactive transitions that occur in the region are determined using data from a scintigram. Both beta and gamma emissions are preferably evaluated. The total radiation absorbed doses are derived for tumor and surrounding tissues. The volumetric data measured from MRI is used to derive the S-values of the tumors using voxel-based simulation (Yoriyaz et al., J Nucl Med 42:662-29, 2001, incorporated herein by reference) to calculate the radiation absorbed doses to the injection sites and surrounding tissues.

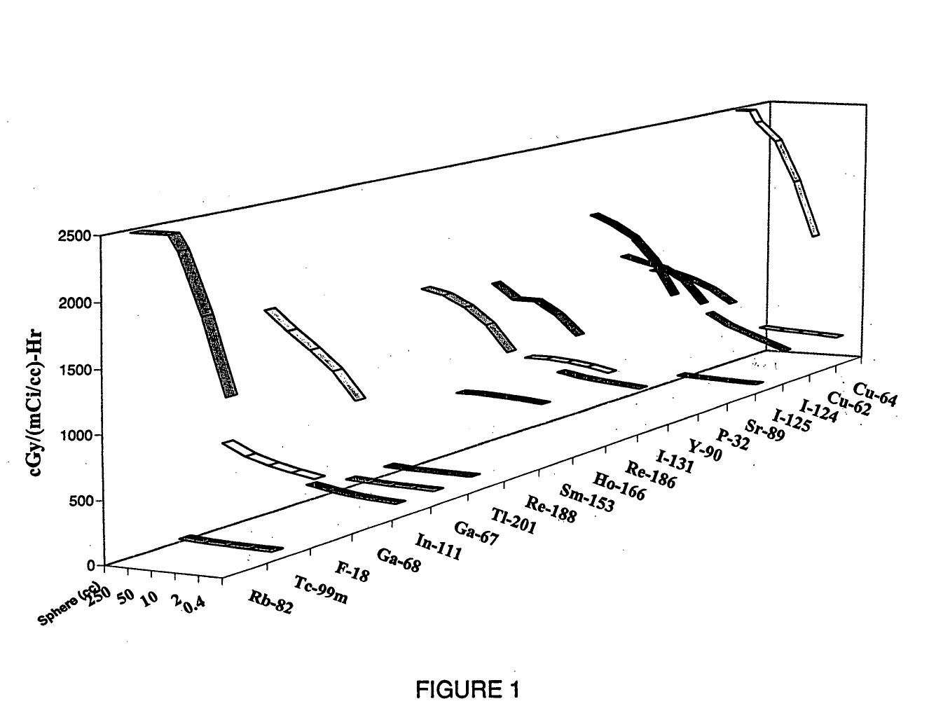

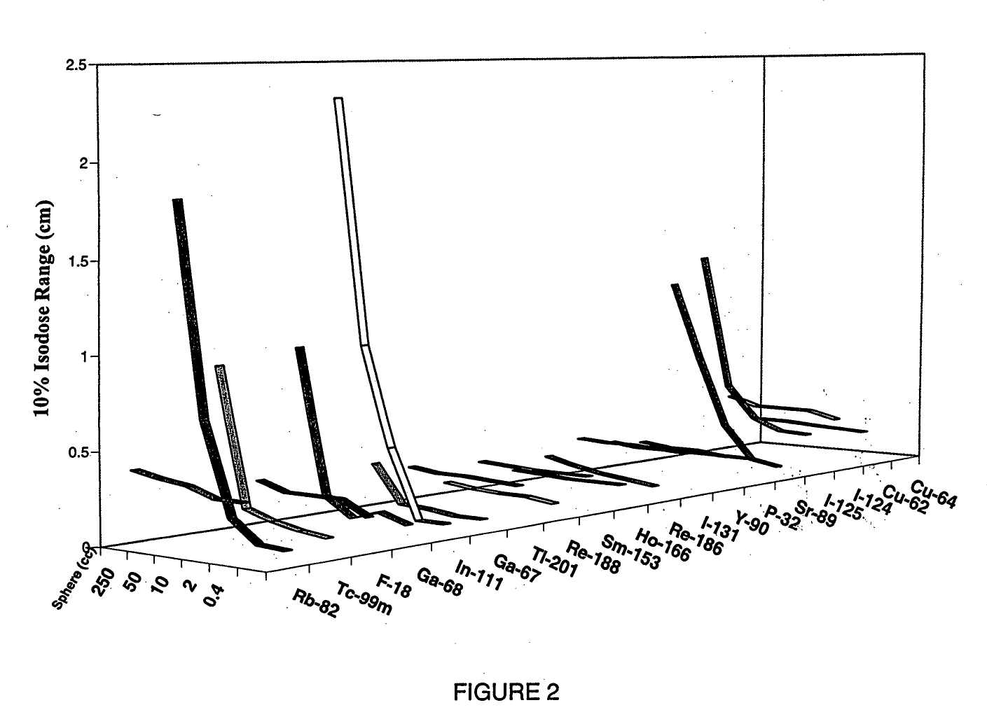

In the literature there are simplistic schemes of dosimetry to estimate radiation doses delivered to an organ or tissues from a point-source or nodules of defined size. One of the inventors has performed Monte Carlo simulations of spheres and shells models filled with 19 different radionuclides, including radionuclides used in the present disclosure. Preliminary results using these simulations have been presented in the abstracts Wong et al., J Nucl Med, 42:243P, 2001; Wong et al., J Nucl Med 43:5, 90P, 2002, incorporated herein by reference. These simulations not only allow the calculation of conventional dosimetry values inside the spheres/shells, but also allow for the calculation of depth dosimetry (radiation does across different distances from the source). Depth dosimetry can be used to establish the efficacy of treatment and safety margins at distances up to 15 cm away from the radiation source. Therefore, these simulations can be adapted by one of skill in the art to calculate the dosimetry of an injected radiopharmaceutical macroaggregate by accurately measuring the volume and radioactivity distribution of the radiopharmaceutical macroaggregate in the subject using MRI/CT and/or Gamma cameras over the course of treatment, as well as depth dosimetry to better establish efficacy and safety margins. Radiation dose sources of the 5 spheres from Monte Carlo simulations of gamma and beta emissions are shown in FIG. 1. FIG. 2 shows the 10% isodose range (i.e., the distance from the sphere where only 10% of the radiation dose from the sphere remains) from simulated depth dosimetry for the 5 spheres.

In preferred embodiments, the radiopharmaceutical macroaggregate is used for locoregional radionuclide therapy of abnormal tissues, for example neoplasms. As used herein, the term “neoplasms” refers to any malignant or benign neoplasms, as well as malignant or benign cancers, solid cancers, and tumors (including any carcinoma, sarcoma, or adenoma). A neoplasm is abnormal tissue that grows by cellular proliferation more rapidly than normal, and can continue to grow after the stimuli that initiated the new growth has ceased. A neoplasm may also have partial or complete lack of structural organization and functional coordination with normal tissue. As used herein, the term “solid cancers” includes but is not limited to the following: bladder tumor, bone tumor, brain tumor, cervical tumor, liver tumor, mammary tumor, ovarian tumor, pituitary tumor, pancreatic tumor, pituitary tumor, prostate tumor, testicular tumor, thyroid tumor, uterine tumor, Wilms' tumor, meninges, adenocarcinoma, adenoma, astrocytoma, Burkitt lymphoma, breast carcinoma, cervical carcinoma, colon carcinoma, kidney carcinoma, liver carcinoma, lung carcinoma, ovarian carcinoma, pancreatic carcinoma, prostate carcinoma, rectal carcinoma, skin carcinoma, melanoma, stomach carcinoma, testis carcinoma, thyroid carcinoma, chondrosarcoma, choriocarcinoma, fibroma, fibrosarcoma, glioblastoma, glioma, hepatoma, histiocytoma, leiomyoblastoma, leiomyosarcoma, lymphoma, liposarcoma cell, medulloblastoma, myeloma, plasmacytoma, neuroblastoma, neuroglioma, osteogenic sarcoma, retinoblastoma, rhabdomyosarcoma, sarcoma, thymoma, and the like.

In other preferred embodiments, the radiopharmaceutical macroaggregate is used for radiosynoviorthesis (Gynter Mödder, Radiosynoviorthesis: Involvement of Nuclear Medicine in Rheumatology and Orthopaedics, 31-54 (Warlich Druck und Verlagsges. Germany, 1995) (2001); incorporated herein by reference). The term “radiosynoviorthesis” as used herein refers to the restoration of the synovia by radiopharmaceutical macroaggregates. Inflammatory diseases such as arthritis are often caused by an inflammatory response of unknown origin in the synovium, or lining, of an afflicted joint. Local application of the radiopharmaceutical macroaggregates is done to influence the synovial process favorably, and as an alternative to surgical synovectomy. Radiosynoviorthesis indications include but are not limited to local therapy of the synovitis; osteoarthritis; rheumatoid diseases such as rheumatoid arthritis, psoriatic arthritis, and Bechterew's disease; villonodular synovitis; haemarthrosis in the haemophiliac; activated arthroses such as knee arthrosis, Baker's cyst, hip arthrosis, condition after total knee replacement, finger polyarthrosis, and rhizarthrosis; dialysis-arthropathies/amyloidosis; and tenosynovitis.

In preferred embodiments, the radiopharmaceutical macroaggregate is injected or punctured into a subject's anesthetized joint (e.g., knee or hip), for the treatment of inflamed synovial tissue. If the initial radiosynoviorthesis treatment is not satisfactory for the subject, for example there is insufficient reduction of pain, local hyperthermia, and/or swelling, the radiosynoviorthesis can be repeated as often as needed. Preferably, however, a second radiosynoviorthesis treatment is performed at least six months after the first treatment.

In preferred embodiments, radiopharmaceutical macroaggregates used for radiosynoviorthesis emit beta particle energy sufficient to penetrate and ablate the synovial tissue, but not so great as to damage underlying articular cartilage or overlying skin. The radiopharmaceutical macroaggregate preferably produces necrosis of abnormal cells in the synovia, as well as a decrease in inflammatory cell proliferation. Additionally, the radiopharmaceutical macroaggregates is preferably sufficient in size to minimize or prevent leakage from the joint, and is biodegradable to prevent induction of granulomatous tissue. Preferably the smaller the joint, the shorter the radiation penetrating distance of the radiopharmaceutical macroaggregate used. The effective dose range for radiosynoviorthesis with radiopharmaceutical macroaggregates may depend on several parameters all of which are familiar to those of skill in the art, including but not limited to the radionuclide used in the co-precipitate, the injected amount, the size if the joint space, synovial thickness, synovial structure, distribution of the radiopharmaceutical macroaggregate in the joint, colloidal absorption into joint fluid, condition of the joint fluid, and inflammatory activity of the synovitis.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

General protocols for generating radiopharmaceuticals by co-precipitating nonradioactive particles with radioactive isotopes to produce a radiopharmaceutical macroaggregate are set forth below. The stock solutions used for the reactions were:

Ferric chloride (FeCl₃) (1 mg Fe/ml): FeCl₃.6H₂O (96.8 mg)+H₂O (20 ml)

Calcium chloride (CaCl₂) (1 mg Ca/ml): CaCl₂ (55.4 mg)+H₂O (20 ml)

Gandolinium chloride (GdCd₃) (1 mg Gd/ml): GdCl₃.6H₂O (47.28 mg)+H₂O (20 ml)

Sodium hydroxide (NaOH) (1.0 N): NaOH (1 g)+H₂O (25 ml)

Ammonium hydroxide (NH₄OH) (20.0%): NH₄OH (5 ml)+H₂O (10 ml)

Phosphate Buffered Saline (PBS): pH=7.0, 7.4, or 8.0

The radionuclides (20-40 μCi/30 μl) were obtained from the following radiopharmacies: ⁶⁷Ga chloride (Mallinkrodt Radiopharmaceuticals); ⁹⁰Y chloride (Nordion), ²⁰¹Tl chloride (Mallinkrodt Radiopharmaceuticals), ⁸⁹Sr chloride (Mallinkrodt Radiopharmaceuticals), ¹¹¹In DTPA (Mallinkrodt Radiopharmaceuticals), ¹¹¹In Cl (Syncor Inc.), ¹⁵³Sm EDTMP (Syncor Inc.), ¹⁶⁶Ho DOTMP (NeoRx, Inc.), ⁶²Cu Cl (Proportional Technology Inc.), ^{99m}Tc pertechnetate (Syncor Inc.), and ¹⁸⁸Re Perrhenate (¹⁸⁸Re generator/University of Missouri).

To generate radiopharmaceutical macroaggregates with Iron as the nonradioactive particle, 500 μl of FeCl₃ was added to 30 μl of any of the above radionuclides (20-40 μCi/30 μl). Next, either 30 μl of NaOH or 10 μl of NH₄OH was added to reach a pH of about 7.0 to 9.0. For stability tests of the radiopharmaceutical macroaggregates generated, a 1:1 dilution with PBS, pH of 7.0, 7.4, or 8.0, was done. To generate radiopharmaceutical macroaggregates with Gadolinium as the nonradioactive particle, 500 μl of GdCl₃ was added to 30 μl of any of the above radionuclides (20-40 μCi/30 μl). Next, either 10 μl of NaOH or 10 μl of NH₄OH was added to reach a pH of about 7.0 to 9.0. For stability tests of the radiopharmaceutical macroaggregates generated, a 1:1 dilution with PBS, pH of 7.0, 7.4, or 8.0, was done. Finally, to generate radiopharmaceutical macroaggregates with Calcium as the nonradioactive particle, 500 μl of CaCl₂ was added to 30 μl of any of the above radionuclides (20-40 μCi/30 μl). Next, either 10 μl of NaOH or 10 μl of NH₄OH was added to reach a pH of about 7.0 to 9.0. For stability tests of the radiopharmaceutical macroaggregates generated, a 1:1 dilution with PBS, pH of 7.0, 7.4, or 8.0, was done.

Co-precipitated radiopharmaceutical macroaggregates generated using the above protocols were separated from remaining soluble radionuclides by centrifuging the reactions from 1500 RPM to 3000 RPM×5 minutes. Alternatively the reactions were filtered using a Millipore Nylone (size: 0.45 μm, diameter: 13 mm) to isolate the radiopharmaceutical macroaggregates. Stability testing of the radiopharmaceutical macroaggregates was done at 0.5, 3.0, 20 and 24 hours in pH 7.4 PBS and in pH 7.0, 7.4, and 8.0 PBS. The radioactivity of the radiopharmaceutical macroaggregates was measured by a calibrated Capintec radiometer in units of μCi. Finally, particle size measurements were calculated using the Submicron Particle Sizer by NICOMP Particle Size Systems (Santa Barbara, Calif., USA).

Tables 2-9 below show the radiochemical yields of the radiopharmaceutical macroaggregates generated using the protocols disclosed above. The radiochemical yield is the fraction of the starting radioactivity (in the initial radionuclide) present in the co-precipitated radiopharmaceutical macroaggregates. The average starting radioactivity of the radionuclides used for generating the radiopharmaceutical macroaggregates was about 50 μCi. Table 2 shows the radiochemical yields after the initial filtration of the co-precipitates but without the addition of any PBS (these radiochemical yields reflect data from at least triplicate samples):

TABLE 2
Radiochemical Yields of Co-precipitates (initial filtration with no PBS):

| Radionuclide | Fe + NaOH | Ca + NaOH | Gd + NaOH | Fe + NH₄OH | Ca + NH₄OH | Gd + NH₄OH |
|---|---|---|---|---|---|---|
| ⁶⁷Ga citrate | 0.18 ± 0.01 | 0.03 ± 0.02 | 0.66 ± 0.21 | 0.95 ± 0.01 | 0.11 ± 0.06 | 0.98 ± 0.00 |
| ⁹⁰Y Cl | 1.00 ± 0.00 | 1.00 ± 0.00 | 1.00 ± 0.00 | 0.94 ± 0.04 | | |
| ⁸⁹Sr Cl | 1.00 ± 0.00 | 0.46 ± 0.00 | 0.25 ± 0.00 | | 0.31 ± 0.00 | 0.26 ± 0.00 |
| ¹⁵³Sm EDTMP | 0.96 ± 0.01 | 0.94 ± 0.00 | 0.96 ± 0.01 | | | |
| ²⁰¹Tl Cl | 0.98 ± 0.00 | 0.08 ± 0.03 | 0.15 ± 0.05 | | 0.07 ± 0.04 | 0.08 ± 0.02 |
| ¹¹¹In DTPA | 0.44 ± 0.05* | 1.00 ± 0.01 | 1.00 ± 0.00 | | | |
| ¹¹¹In Cl | 0.98 ± 0.02 | 0.98 ± 0.01 | 0.97 ± 0.01 | | | |
| ¹⁸⁸Re Perrhenate | | | | 0.16 ± 0.01 | 0.17 ± 0.01 | 0.18 ± 0.01 |
| ¹⁶⁶Ho DOTMP | 0.44 ± 0.03 | 0.96 ± 0.01 | 0.94 ± 0.02 | 0.97 ± 0.03 | | |
| ^{99m}TcO₄ | 0.94 ± 0.02 | 0.96 ± 0.01 | 0.81 ± 0.01 | | | |
| ⁶²Cu | 0.99 ± 0.00 | 0.74 ± 0.07 | 0.94 ± 0.00 | | | |

*indicates double portions of NaOH were used to precipitate the radiopharmaceutical macroaggregates (pH > 10), which improved the yield to 0.98 ± 0.01.

As shown in Table 2, the co-precipitates of ⁹⁰Y, ¹⁵³Sm, and ¹¹¹In generated high radiochemical yields regardless of the metal used to co-precipitate the radionuclides. In contrast, the radiochemical yields for the co-precipitation of other radionuclides, for example ²⁰¹Tl and ¹⁶⁶Ho, were highly variable depending on the metal used. Table 3 presents the radiochemical yields for reactions at both a high temperature (90° C.) and a low temperature (0° C.). As shown by the data, temperature generally did not significantly effect the radiochemical yields, other than for the ¹¹¹In-Gd co-precipitate:

TABLE 3

| | Fe + NaOH | Ca + NaOH | Gd + NaOH | Fe + NaOH | Ca + NaOH | Gd + NaOH |
|---|---|---|---|---|---|---|
| Radionuclide | 90° C. | 90° C. | 90° C. | 0° C. | 0° C. | 0° C. |
| ¹¹¹In DTPA | 0.97 ± 0.01* | 0.99 ± 0.01 | 0.85 ± 0.01 | 0.92 ± 0.01 | 0.99 ± 0.00 | 0.29 ± 0.04 |
| ¹¹¹In Cl | 0.96 ± 0.01 | 0.95 ± 0.02 | 0.95 ± 0.01 | 0.96 ± 0.00 | 0.97 ± 0.01 | 0.93 ± 0.01 |

*indicates double portions of NaOH were used to precipitate the radiopharmaceutical macroaggregates (pH > 10).

Tables 4 and 5 show the radiochemical yields of the co-precipitates after addition of PBS. As shown by the data, the pH of the PBS added to the radiopharmaceutical macroaggregates did not appear to effect the radiochemical yields of the reaction:

TABLE 4

| | Fe + NaOH | Ca + NaOH | Gd + NaOH | Fe + NaOH | Ca + NaOH | Gd + NaOH | Fe + NaOH | Ca + NaOH | Gd + NaOH |
|---|---|---|---|---|---|---|---|---|---|
| Radionuclide | pH7.0 | pH7.0 | pH7.0 | pH7.4 | pH7.4 | pH7.4 | pH8.0 | pH8.0 | pH8.0 |
| ⁹⁰Y Cl | 1.00 ± 0.00 | 0.95 ± 0.09 | 1.00 ± 0.00 | 1.00 ± 0.00 | 1.00 ± 0.01 | 1.00 ± 0.00 | 1.00 ± 0.00 | 0.93 ± 0.06 | 0.99 ± 0.02 |
| ⁸⁹Sr CL | 0.96 ± 0.07 | | | 0.94 ± 0.01 | | | 0.91 ± 0.02 | | |
| ¹⁵³Sm | 0.98 ± 0.01 | 0.99 ± 0.00 | 0.98 ± 0.00 | 0.98 ± 0.00 | 0.99 ± 0.00 | 0.97 ± 0.00 | 0.98 ± 0.00 | 0.99 ± 0.00 | 0.97 ± 0.00 |
| EDTMP | | | | | | | | | |
| ²⁰¹Tl Cl | 0.96 ± 0.00 | | | 0.97 ± 0.00 | | | 0.97 ± 0.00 | | |
| ¹¹¹In DTPA | 0.96 ± 0.01* | 1.00 ± 0.00 | 0.48 ± 0.08 | 0.94 ± 0.01* | 1.00 ± 0.00 | 0.69 ± 0.14 | 0.98 ± 0.01* | 1.00 ± 0.00 | 0.48 ± 0.04 |
| ¹¹¹In Cl | 0.94 ± 0.03 | 0.93 ± 0.02 | 0.96 ± 0.01 | 0.96 ± 0.01 | 0.95 ± 0.02 | 0.96 ± 0.02 | 0.96 ± 0.03 | 0.94 ± 0.02 | 0.97 ± 0.01 |
| ¹⁶⁶Ho | | 0.97 ± 0.00 | 0.47 ± 0.02 | | 0.98 ± 0.01 | 0.51 ± 0.02 | | 0.98 ± 0.03 | 0.48 ± 0.01 |
| DOTMP | | | | | | | | | |

*indicates double portions of NaOH were used to precipitate the radiopharmaceutical macroaggregates (pH > 10).

TABLE 5

| | Fe + NH₄OH | Gd + NH₄OH | Fe + NH₄OH | Gd + NH₄OH | Fe + NH₄OH | Gd + NH₄OH |
|---|---|---|---|---|---|---|
| Radionuclide | pH7.0 | pH7.0 | pH7.4 | pH7.4 | pH8.0 | pH8.0 |
| ⁶⁷Ga citrate | 0.99 ± 0.00 | 0.63 ± 0.07 | 0.99 ± 0.00 | 0.66 ± 0.05 | 0.99 ± 0.00 | 0.56 ± 0.02 |
| ¹⁶⁶Ho | 0.78 ± 0.02 | | 0.77 ± 0.02 | | 0.72 ± 0.01 | |
| DOTMP | | | | | | |

Tables 6-9 show the stability of various radiopharmaceutical macroaggregates over a period of 24 hours by monitoring the radiochemical yields at various timepoints. Generally the radiopharmaceutical macroaggregates demonstrated remarkable stability over the 24 hour time period:

TABLE 6
Fe + NaOH Time Stability in pH 7.4 PBS

| Radionuclide | 0.5 H | 3 H | 20 H | 24 H |
|---|---|---|---|---|
| ⁹⁰Y Cl | 0.98 ± 0.00 | | 1.00 ± 0.00 | 1.00 ± 0.00 |
| ⁸⁹Sr Cl | 1.00 ± 0.00 | | | 0.85 ± 0.02 |
| ¹⁵³Sm EDTMP | 0.99 ± 0.01 | 0.98 ± 0.01 | 1.00 ± 0.01 | 0.99 ± 0.00 |
| ²⁰¹Tl Cl | 0.97 ± 0.00 | 0.97 ± 0.01 | 0.97 ± 0.01 | 0.97 ± 0.00 |
| ¹¹¹In DTPA | 0.97 ± 0.01 | 0.99 ± 0.01 | 0.92 ± 0.01 | 0.95 ± 0.03 |
| ¹¹¹In Cl | 0.95 ± 0.02 | 0.97 ± 0.01 | 0.96 ± 0.03 | 0.97 ± 0.02 |

TABLE 7
Ca + NaOH Time Stability in pH 7.4 PBS

| Radionuclide | 0.5 H | 3 H | 20 H | 24 H |
|---|---|---|---|---|
| ¹⁵³Sm EDTMP | 0.99 ± 0.00 | 0.99 ± 0.01 | 0.99 ± 0.01 | 0.99 ± 0.01 |
| ¹¹¹In DTPA | 1.00 + 0.00 | 1.00 + 0.00 | 0.92 ± 0.01 | 0.95 ± 0.01 |
| ¹¹¹In Cl | 0.96 ± 0.02 | 0.97 ± 0.02 | 0.96 ± 0.01 | 0.96 ± 0.01 |
| ¹⁶⁶Ho DOTMP | 0.97 ± 0.01 | 0.95 ± 0.02 | 0.95 ± 0.02 | 0.96 ± 0.03 |

TABLE 8
Gd + NaOH Time Stability in pH 7.4 PBS

| Radionuclide | 0.5 H | 3 H | 20 H | 24 H |
|---|---|---|---|---|
| ¹⁵³Sm EDTMP | 0.97 ± 0.00 | 0.97 ± 0.01 | 0.99 ± 0.01 | 0.99 ± 0.01 |
| ¹¹¹In DTPA | 0.57 ± 0.09 | 0.47 ± 0.26 | 0.50 ± 0.03 | 0.56 ± 0.05 |
| ¹¹¹In Cl | 0.96 ± 0.01 | 0.97 ± 0.01 | 0.96 ± 0.01 | 0.96 ± 0.01 |
| ¹⁶⁶Ho DOTMP | 0.45 ± 0.03 | 0.37 ± 0.03 | 0.14 ± 0.02 | 0.14 ± 0.04 |

TABLE 9
Fe + NH₄OH Time Stability in pH 7.4 PBS

| Radionuclide | 0 H | 0.5 H | 3 H | 20 H | 24 H |
|---|---|---|---|---|---|
| ⁶⁷Ga citrate | 0.82 ± 0.05 | 0.78 ± 0.01 | 0.74 ± 0.03 | 0.76 ± 0.05 | 0.78 ± 0.11 |
| ¹⁶⁶Ho DOTMP | | 0.69 ± 0.01 | 0.61 ± 0.02 | 0.47 ± 0.03 | 0.40 ± 0.01 |

Tables 10-13 below show the particle sizes of the various radiopharmaceutical macroaggregates generated using the protocols disclosed above (but without the inclusion of a radionuclide in the radiopharmaceutical macroaggregate), including measurements by volume and by number (Berger et al., Int J Pharmaceutics 223:55, 2001, incorporated herein by reference). No radionuclide was present in the radiopharmaceutical macroaggregates used for particle size measurements because only infinitesimal amounts of radionuclides are present in the radiopharmaceutical macroaggregates (approximately <1/10⁶). The radiopharmaceutical macroaggregates were suspended in 20% gelatin to calculate particle sizes, and the measurements are shown in nanometers. In Table 10, the more important measurement is the one based on volume because the volume is proportional to the radioactivity levels of the radiopharmaceutical macroaggregates. For example, one 10 micron particle will deliver much more desired radioactivity than 100 particles of 0.1 micron. The data in Tables 11-13 demonstrate that factors such as dilution, pH, and centrifugation greatly effect the sizes of the radiopharmaceutical macroaggregates.

TABLE 10
A. Repeated Sampling

| | Sample | | | | | | |
|---|---|---|---|---|---|---|---|
| | Fe | | | | | | |
| | 1 | 2 | 3 | 4 | 5 | 6 | 7 |
| Volume | | | | | | | |
| Mean | 11284.9 | 58133.6 | 32881.5 | 22076.8 | 25616.3 | 29866.6 | 19741 |
| Deviation | 17423.8 | 267874.2 | 34490.9 | 25121.1 | 39078.6 | 44179.2 | 28520.7 |
| Chi | 0.071 | 0.957 | 0.943 | 1.834 | 0.523 | 0.187 | 108.198 |
| Squared | | | | | | | |
| Number | | | | | | | |
| Mean | 672.7 | 2704.1 | 3771.3 | 2492.5 | 2738.8 | | 1252.6 |
| Deviation | 1038.6 | 13643.3 | 5797.3 | 13355.3 | 3591.6 | | 1851.3 |
| Chi | 0.071 | 0.922 | 0.494 | 4.609 | 0.096 | | 114.887 |
| Squared | | | | | | | |
| Mean/Vol | 29229.733 | | | | | | |
| Mean/No | 2104.5875 | | | | | | |

| | Sample | | | | | |
|---|---|---|---|---|---|---|
| | Fe | | Ca | | | Gd |
| | 8 | 9 | 1 | 2 | 3 | 1 |
| Volume | | | | | | |
| Mean | 33990.2 | 29476.7 | 14123.9 | 8040.3 | 5036.5 | 22749.9 |
| Deviation | 103369.5 | 50394.4 | 16445.5 | 7762.7 | 4342.6 | 37718.7 |
| Chi | 1.912 | 7.661 | 0.422 | 0.415 | 0.216 | 17.667 |
| Squared | | | | | | |
| Number | | | | | | |
| Mean | 1567 | 1637.7 | 880.9 | | | 1281.3 |
| Deviation | 4637.4 | 2861.1 | 804.8 | | | 2124.4 |
| Chi | 2.916 | 7.57 | 0.645 | | | 17.667 |
| Squared | | | | | | |
| Mean/Vol | | | 9066.9 | | | 22749.9 |
| Mean/No | | | 880.9 | | | 1281.3 |

TABLE 11
B. Effects of Dilutions
Fe

| | | | | | |
|---|---|---|---|---|---|
| Dilution | 2 | 4 | | 8 | 16 |
| ratios | | | | | |
| NO | 1 | 1 | 2 | 1 | 1 |
| Volume | | | | | |
| Mean | 60739.3 | 22634.5 | 26515.5 | 16046.9 | 12858.5 |
| Deviation | 101427.7 | 31097.3 | 38719.7 | 19072.1 | 11080.6 |
| Chi | 16.428 | 0.366 | 1.194 | 2.074 | 0.692 |
| Squared | | | | | |
| Number | | | | | |
| Mean | 3403.1 | 1619.4 | | 4386.4 | 1887.4 |
| Deviation | 5682.8 | 2299.6 | | 30339.5 | 1626.4 |
| Chi | 16.428 | 0.524 | | 129.609 | 0.692 |
| Squared | | | | | |
| Mean/Vol | 60739.3 | 22634.5 | | 16046.9 | |
| Mean/No | 3403.1 | 1619.4 | | 4386.4 | |

TABLE 12
C. Effects of pH on Size

| Fe | PH7.0 | | PH7.4 | | PH8.0 | |
|---|---|---|---|---|---|---|
| sample | 1 | 2 | 1 | 2 | 1 | 2 |
| Volume | | | | | | |
| Mean | 14692.4 | 12334.9 | 21295.3 | 21036.8 | 18131.1 | 12590.8 |
| Deviation | 17262.8 | 11872.7 | 33818.3 | 29198.3 | 35362.5 | 23344.4 |
| Chi Squared | 1.878 | 3.395 | 5.036 | 3 | 302.634 | 623.581 |
| Number | | | | | | |
| Mean | 1137.1 | | 1240 | | 1000.4 | 683 |
| Deviation | 1328.2 | | 1969.2 | | 1990.2 | 1276.9 |
| Chi Squared | 1.785 | | 5.036 | | 296.58 | 558.793 |
| Mean/Vol | 13513.65 | | 21166.05 | | 15360.95 | |
| Mean/No | 1137.1 | | 1240 | | 1000.4 | |

TABLE 13
D. Effects of Centrifugation x3

| | Fe | Ca | Ca | Gd |
|---|---|---|---|---|
| NO | 1 | 1 | 2 | 1 |
| Volume | | | | |
| Mean | 32368.8 | 14792.8 | 17575.5 | 19650.9 |
| Deviation | 52251.4 | 10672.9 | 14511.6 | 30424 |
| Chi Squared | 0.239 | 0.97 | 0.563 | 7.272 |
| Number | | | | |
| Mean | 1945.3 | 3324.3 | 2850.1 | 1168.6 |
| Deviation | 2517.3 | 2398.4 | 2353.3 | 1809.3 |
| Chi Squared | 0.494 | 0.967 | 0.563 | 7.272 |
| mean/Vol | 32368.8 | 16184.15 | | 19650.9 |
| mean/No | 1945.3 | 3324.3 | | 1168.6 |

### EXAMPLE 2

Radiopharmaceutical macroaggregate can also be generated by co-precipitating two radionuclide isotopes with one non-radioactive metal. To generate these double-labeled radiopharmaceutical macroaggregate the same stock solutions set forth in Example 1 were generated. The radionuclides used were ⁶⁷Ga citrate, ⁹⁰Y Cl, ¹¹¹In Cl, and ^{99m}TcO₄ at a concentration of 10 μCi/ml.

To generate the double-labeled radiopharmaceutical macroaggregates, 500 μl of FeCl₃, GdCl₃, CaCl₂ was combined with 50 μl of ⁶⁷Ga citrate, ¹¹¹In DTPA, or ^{99m}TcO₄, and 50 μl of ⁹⁰Y Cl. Next, either 10 μl or 30 μl of NaOH, or 10 μl of NH₄OH, was added to the reaction, and the pH was adjusted to about 7.0 to 9.0. The co-precipitated radiopharmaceutical macroaggregates generated using the above protocol were separated from remaining soluble radionuclides by centrifuging the reactions at 3000 RPM×5 minutes, and then washed twice with 1 ml PBS, followed by measurements using the r-counter. For stability tests of the double-labeled radiopharmaceutical macroaggregates generated, the radiopharmaceutical macroaggregates were washed twice with 1 ml PBS, and resuspended in 1 ml PBS. The radiochemical yields of the radiopharmaceutical macroaggregates were monitored over a 24 to 96 hour period by a gamma-counter.

Tables 14-16 show the radiochemical yields for the double-labeled radiopharmaceutical macroaggregates generated. Table 14 shows the double-labeling and stability for ⁹⁰Y-Fe-^{99m}Tc, ⁹⁰Y-Ca-^{99m}Tc, and ⁹⁰Y-Gd-^{99m}Tc radiopharmaceutical macroaggregates. Table 15 shows the double-labeling and stability for ⁹⁰Y-Fe-⁶⁷Ga, ⁹⁰Y-Ca-⁶⁷Ga, and ⁹⁰Y-Gd-⁶⁷Ga radiopharmaceutical macroaggregates. And Table 16 shows the double-labeling and stability for ⁹⁰Y-Fe-¹¹¹In, ⁹⁰Y-Ca-¹¹¹In, and ⁹⁰Y-Gd-¹¹¹In radiopharmaceutical macroaggregates.

TABLE 14
^{99m}Tc + ⁹⁰Y double-labeling and stability

| | 0 H | | 3 H | | 24 H | |
|---|---|---|---|---|---|---|
| | Tc % | Y % | Tc % | Y % | Tc % | Y % |
| | in Mix | in Mix | in Mix | in Mix | in Mix | in Mix |
| Fe | 82.3 | 70.7 | 90.7 | 61.2 | 87.8 | 57.8 |
| Ca | 90.9 | 94.2 | 94.1 | 75.1 | 91.2 | 87.7 |
| Gd | 35.9 | 22.9 | 90.7 | 61.2 | 88.6 | 63.5 |
| Recovery in | 94.2 | 91.3 | 89.0 | 108.0 | 89.0 | 108.0 |
| Mixture of Stds | | | | | | |

TABLE 15
⁶⁷Ga + ⁹⁰Y double-labeling and stability

| | 0 H | | 24 H | |
|---|---|---|---|---|
| | Y % in Mix | Ga % in Mix | Y % in Mix | Ga % in Mix |
| Fe | 90.6 | 97.5 | 92.3 | 86.9 |
| Recovery in | 104.1 | 96.4 | 104.1 | 96.4 |
| Mixture of Stds | | | | |

TABLE 16
¹¹¹In + ⁹⁰Y double-labeling and stability

| | 0 H | | 3 H | | 24 H | | 48 H | | 96 H | |
|---|---|---|---|---|---|---|---|---|---|---|
| | Y % in | In % in | Y % in | In % in | Y % in | In % in | Y % in | In % in | Y % in | In % in |
| | Mix | Mix | Mix | Mix | Mix | Mix | Mix | Mix | Mix | Mix |
| Fe | 62.5 | 88.2 | 55.0 | 91.3 | 51.7 | 84.7 | 43.0 | 83.0 | 45.7 | 85.9 |
| Ca | 76.1 | 81.2 | 86.4 | 90.5 | 60.7 | 69.4 | 70.9 | 80.0 | 66.3 | 77.0 |
| Gd | 73.8 | 93.6 | 44.7 | 78.6 | 48.3 | 73.9 | 62.9 | 83.4 | 47.8 | 87.8 |
| Recovery | 109.4 | 101.8 | 94.6 | 101.1 | 94.6 | 101.1 | 94.6 | 101.1 | 94.6 | 101.1 |
| in Mixture | | | | | | | | | | |
| of Stds | | | | | | | | | | |

Determining valid radiochemical yield measurements of double-labeled radiopharmaceutical macroaggregates is difficult because two different radioactive isotopes are present in a single radiopharmaceutical macroaggregate. A Hewlett-Packard Cobra-II gamma counter was used to measure the radioactivity of the radiopharmaceutical macroaggregates. The radiation detection window was set narrowly but distinctly apart for the two isotopes. Note that the typical default settings for the isotopes may not work. For measurements, the standards for each isotope, as well as a mixture of identical amounts of the standards for each isotope, were placed under the gamma camera for simultaneous measurements using at least two windows. The measurement methods were validated by recovery of both isotopes in the 1.000+0.125 range (Recovery in Mixture of Stds value in above Tables 14-16).

### EXAMPLE 3

Another method for producing co-precipitates of paramagnetic or nonparamagnetic metals with radionuclides involves the use of Phytate (C₆H₁₂O₁₈P₆, or P). To generate these radiopharmaceutical macroaggregates, 50 μl of an aqueous solution of sodium phytate (50 mg/ml) was mixed with about 2-100 μCi (typically 50 μCi) of a radionuclide (obtained from the sources set forth in Example 1), with or without 50 μl of tin chloride (SnCl₂, 5 mg/ml). Next, CaCl₂, FeCl₃, or GdCl₃ solutions were added to the reaction, and after 5 minutes the mixture was collected by centrifuging the reactions at 3000 RPM×5 minutes. The radiochemical yield for each of the radiopharmaceutical macroaggregates generated was measured, and stability tests in PBS at pH 7.4 over 24 hours were also performed as set forth in Example 1.

These radiopharmaceutical macroaggregates are co-precipitates of a non-radioactive metal (M) with a radionuclide cation (C) or a radionuclide anion (A), and P. The M used were Ca, Fe, or Gd; the C used were ⁶⁷Ga citrate, ⁹⁰Y Cl, ¹²³I, ²⁰¹Tl Cl, ⁶²Cu Cl, or ¹¹¹In Cl; and the A used were ¹⁸⁸Re Perrhenate or ^{99m}TcO₄. The ^{99m}Tc co-precipitates demonstrated good stability over 24 hours.

Tables 17-20 show the radiochemical yields for the radiopharmaceutical macroaggregates generated with Phytate using the protocol disclosed above, as well as the stability of these radiopharmaceutical macroaggregates in PBS pH 7.4 over a 24 hour time period. As shown in Table 17, high radiochemical yields were found for co-precipitates of ⁶⁷Ga, ⁹⁰Y, ¹¹¹In, or ^{99m}Tc with non-radioactive Ca, Fe, or Gd. Lower radiochemical yields were found with co-precipitates of ²⁰¹Tl, ⁶²Cu, or ¹⁸⁸Re and Ca, Fe, or Gd. The precipitated radiopharmaceutical macroaggregates varied in size from 6-40 microns. No precipitation was found with ¹²³I.

TABLE 17

| Radionuclide | Ca | Fe | Gd |
|---|---|---|---|
| ^{99m}TcO₄ | 0.90 ± 0.06 | 0.91 ± 0.03 | 0.90 ± 0.01 |
| ⁶⁷Ga citrate | 0.98 ± 0.03 | 0.84 ± 0.05 | 0.97 ± 0.01 |
| ⁹⁰Y Cl | 0.86 ± 0.01 | 0.68 ± 0.09 | 0.71 ± 0.05 |
| ¹²³I | 0.08 ± 0.01 | 0.23 ± 0.09 | 0.09 ± 0.02 |
| ¹⁸⁸Re Perrhenate | 0.24 ± 0.01 | 0.20 ± 0.01 | 0.23 ± 0.01 |
| ²⁰¹Tl Cl | 0.27 ± 0.01 | 0.34 ± 0.01 | 0.18 ± 0.01 |
| ¹¹¹In Cl | 0.88 ± 0.02 | 0.80 ± 0.03 | 0.89 ± 0.02 |
| ⁶²Cu Cl | 0.44 ± 0.01 | 0.27 ± 0.04 | 0.31 ± 0.02 |

TABLE 18
Fe Time Stability in pH 7.4 PBS

| Radionuclide | 0.5 H | 3 H | 20 H | 24 H |
|---|---|---|---|---|
| ^{99m}TC | 0.85 ± 0.01 | 0.79 ± 0.03 | 0.76 ± 0.03 | 0.78 ± 0.04 |
| ⁹⁰Y Cl | 0.68 ± 0.04 | 0.76 ± 0.05 | 0.75 ± 0.03 | 0.74 ± 0.05 |
| ¹¹¹In Cl | 0.82 ± 0.04 | 0.82 ± 0.04 | 0.79 ± 0.03 | 0.79 ± 0.03 |
| ⁶⁷Ga Citrate | 0.75 ± 0.09 | 0.69 ± 0.01 | 0.68 ± 0.02 | 0.66 ± 0.09 |

TABLE 19
Ca Time Stability in pH 7.4 PBS

| Radionuclide | 0.5 H | 3 H | 20 H | 24 H |
|---|---|---|---|---|
| ^{99m}TC | 0.77 ± 0.03 | 0.79 ± 0.01 | 0.74 ± 0.05 | 0.68 ± 0.05 |
| ⁹⁰Y Cl | 0.92 ± 0.04 | 0.93 ± 0.04 | 0.91 ± 0.02 | 0.88 ± 0.03 |
| ¹¹¹In Cl | 0.91 ± 0.01 | 0.91 ± 0.01 | 0.90 ± 0.01 | 0.89 ± 0.03 |
| ⁶⁷Ga Citrate | 0.75 ± 0.01 | 0.71 ± 0.07 | 0.78 ± 0.03 | 0.70 ± 0.03 |

TABLE 20
Gd Time Stability in pH 7.4 PBS

| Radionuclide | 0.5 H | 3 H | 20 H | 24 H |
|---|---|---|---|---|
| ⁹⁰Y Cl | 0.90 ± 0.02 | 0.85 ± 0.02 | 0.73 ± 0.07 | 0.77 ± 0.07 |
| ¹¹¹In Cl | 0.89 ± 0.02 | 0.88 ± 0.03 | 0.89 ± 0.04 | 0.87 ± 0.01 |
| ⁶⁷Ga Citrate | 0.87 ± 0.01 | 0.85 ± 0.02 | 0.83 ± 0.02 | 0.90 ± 0.02 |

Table 21 shows the radiochemical yields and stability for radiopharmaceutical macroaggregates that include ⁹⁰Y and Fe generated both with and without Phytate using the protocols disclosed above and in Example 1, except that the starting radioactivity of the ⁹⁰Y Cl used for generating these radiopharmaceutical macroaggregates was about 10 mCi. This high dose experiment did appear to improve the radiochemical yield and stability of the ⁹⁰Y-Fe-P radiopharmaceutical macroaggregate.

TABLE 21

| Radionuclide | 0 H | 24 H | 72 H |
|---|---|---|---|
| ⁹⁰Y—Fe | 0.94 ± 0.03 | 0.94 ± 0.03 | 0.92 ± 0.09 |
| ⁹⁰Y—Fe-P | 0.37 ± 0.03 | 0.99 ± 0.01 | 0.92 ± 0.05 |

Finally, double-labeled radiopharmaceutical macroaggregates with Phytate were produced using mixed anion-cation co-precipitates, as well as cation-cation co-precipitates. To generate these radiopharmaceutical macroaggregates, 50 μl of ⁶⁷Ga citrate, ¹¹¹In DTPA, or ^{99m}TcO₄, and 50 μl of ⁹⁰Y Cl (approximately 50 μCi of each radionuclide), were added to 50 μl of phytic acid (50 mg/ml), with or without 50 μl of SnCl₂ (5 mg/ml). The concentration of all radionuclides used was 10 μCi/ml. The reaction was allowed to mix for 10 minutes, and next 50 μl of 0.5 M solutions of CaCl₂, FeCl₃, or GdCl₃ were added to the reaction and mixed for 2 minutes. The double-labeled radiopharmaceutical macroaggregates were separated from remaining soluble radionuclides by centrifuging the reactions at 3000 RPM×5 minutes, and then washed twice with 1 ml PBS, followed by measurements using a gamma counter. For stability tests of the double-labeled radiopharmaceutical macroaggregates generated, the radiopharmaceutical macroaggregates were washed twice with 1 ml PBS, and resuspended in 1 ml PBS, pH 7.4. The radiochemical yields of the radiopharmaceutical macroaggregates were monitored over a 24 to 96 hour period by a gamma-counter.

Tables 22-24 show the radiochemical yields for the double-labeled radiopharmaceutical macroaggregates generated with Phytate using the protocol disclosed above, as well as the stability for one of these radiopharmaceutical macroaggregates in PBS pH 7.4 over a 96 hour time period. The radiochemical yields of the double-labeled radiopharmaceutical macroaggregates with Phytate generated were measured as described in Example 2.

TABLE 22
^{99m}Tc + ⁹⁰Y (Phytate) double-labeling and stability

| | 0 H | |
|---|---|---|
| | Tc % in Mix | Y % in Mix |
| Ca | 97.4 | 75.6 |
| Fe | 97.1 | 49.3 |
| Gd | 97.5 | 46.6 |
| Recovery in Mixture of | 89.0 | 108.0 |
| Stds | | |

TABLE 23
⁶⁷Ga + ⁹⁰Y (Phytate) double-labeling and stability

| | 0 H | |
|---|---|---|
| | Y % in Mix | Ga % in Mix |
| Ca | 38.1 | 84.3 |
| Fe | 47.8 | 88.2 |
| Gd | 50.0 | 96.1 |
| Recovery in Mixture of | 104.1 | 96.4 |
| Stds | | |

TABLE 24
¹¹¹In + ⁹⁰Y (Phytate) double-labeling and stability

| | 0 H | | 3 H | | 24 H | | 48 H | | 96 H | |
|---|---|---|---|---|---|---|---|---|---|---|
| | Y % | In % | Y % | In % | Y % | In % | Y % | In % | Y % | In % |
| | in | in | in | in | in | in | in | in | in | in |
| | Mix | Mix | Mix | Mix | Mix | Mix | Mix | Mix | Mix | Mix |
| Ca | 79.3 | 92.5 | 61.2 | 85.6 | 51.1 | 88.1 | 92.5 | 83.4 | 68.4 | 82.2 |
| Fe | 19.1 | 49.2 | 33.3 | 49.2 | 20.4 | 54.9 | 34.6 | 53.4 | 21.7 | 50.8 |
| Gd | 30.3 | 90.2 | 35.8 | 83.5 | 27.7 | 82.6 | 45.2 | 82.1 | 26.9 | 81.6 |
| Recovery | 109.4 | 101.8 | 94.6 | 101.1 | 94.6 | 101.1 | 94.6 | 101.1 | 94.6 | 101.1 |
| in Mixture | | | | | | | | | | |
| of Stds | | | | | | | | | | |

### EXAMPLE 4

General protocols for generating radiopharmaceuticals by the mechanism of adsorption (of radioactive isotopes by nonradioactive particles to produce a radiopharmaceutical macroaggregate are set forth below. The stock solutions used for the reactions were:

Ferric chloride (FeCl₃) (1 mg Fe/ml): FeCl₃.6H₂O (96.8 mg)+H₂O (20 ml)

Calcium chloride (CaCl₂) (1 mg Ca/ml): CaCl₂ (55.4 mg)+H₂O (20 ml)

Gandolinium chloride (GdCd₃) (1 mg Gd/ml): GdCl₃.6H₂O (47.28 mg)+H₂O (20 ml)

Sodium hydroxide (NaOH) (1.0 N): NaOH (1 g)+H₂O (25 ml)

Ammonium hydroxide (NH₄OH) (20.0%): NH₄OH (5 ml)+H₂O (10 ml)

Phosphate Buffered Saline (PBS): pH=7.0, 7.4, or 8.0

Each stock of 1 mg/ml of FeCl₃, CaCl₂, and GdCl₃ was made fresh and titrated with NaOH or NH₄OH (for FeCl₃) to a pH of 8.0. The precipitates formed by this reaction were washed with 0.1 mM PBS, and centrifuged at 3000 rpm for 5 minutes. Next, a small volume of a radionuclide (1-2 μCi), was added to the precipitates for 5-10 minutes. The precipitates were again washed with PBS and centrifuged two times to remove any remaining soluble radionuclides at 3000 rpm for 5 minutes. The radiochemical yields of all the radiopharmaceutical macroaggregates described below in this example were measured by a gamma-counter and compared with standards.

Table 25 below show the radiochemical yields of the radiopharmaceutical macroaggregates generated using the protocols disclosed above. The average starting radioactivity of the radionuclides used for generating the radiopharmaceutical macroaggregates was about 1-2 μCi:

TABLE 25
Radiochemical Yields by Adsorption

| Radionuclide | Fe + NH₄OH | Ca + NaOH | Gd + NaOH |
|---|---|---|---|
| ^{99m}Tc pertechnetate | 4.46 ± 1.30 | | |
| ¹³¹I NaI | 2.91 ± 0.24 | | |
| ¹¹¹In DTPA | 4.52 ± 0.15 | | |
| ⁶⁷Ga citrate | 68.09 ± 1.14 | 13.84 ± 1.89 | 85.20 ± 2.00 |
| ²⁰¹Tl Cl | 90.71 ± 0.17 | 48.37 ± 0.38 | 44.09 ± 3.00 |
| ¹⁵³Sm EDTMP | 46.08 ± 4.11 | 76.40 ± 6.39 | 95.15 ± 3.29 |
| ⁸⁹Sr Cl | 80.58 ± 12.80 | 9.90 ± 1.73 | 12.08 ± 2.23 |
| ⁹⁰Y Cl | 27.8 ± 1.8 | 16.9 ± 14.5 | 86.3 ± 2.0 |
| ¹¹¹In Cl | 40.4 ± 1.8 | 69.1 ± 8.3 | 86.8 ± 6.1 |

As shown in Table 25, the radiopharmaceutical macroaggregates generated by adsorption of ²⁰¹Tl and ¹⁵³Sm had high radiochemical yields regardless of the metal used. In contrast, the radiochemical yields for the radiopharmaceutical macroaggregates of other radionuclides, for example ⁶⁷Ga and ⁸⁹Sr, were highly variable depending on the metal used.

To determine whether a delay in the addition of the radionuclide to nonradioactive particles affected the adsorption efficiency, “aged” Fe radiopharmaceutical macroaggregates were compared to radiopharmaceutical macroaggregates generated by the method set forth above. The first set of radiopharmaceutical macroaggregates was generated by immediately adding the radionuclide to the Fe particles, as set forth above. The second set of radiopharmaceutical macroaggregates was generated by first generating the Fe macroaggregates, adding the radionuclide after 24 hours had passed, and isolating the radiopharmaceutical macroaggregates as set forth above. Table 26 shows the results of this experiment, and demonstrates that the delay in adding the radionuclide did not significantly affect the radiochemical yields:

TABLE 26
Radiochemical Yields of “Aged” Radiopharmaceutical
Macroaggregates
% yield of “immediate” radiopharmaceutical macroaggregates

| | |
|---|---|
| ⁶⁷Ga citrate | 68.09 ± 1.14 |
| ²⁰¹Tl Cl | 90.71 ± 0.17 |

% yield of “aged” radiopharmaceutical macroaggregates

| | |
|---|---|
| ⁶⁷Ga citrate | 64.01 ± 1.05 |
| ²⁰¹Tl Cl | 95.56 ± 0.65 |

The stability of various radiopharmaceutical macroaggregates generated by the mechanism of adsorption was also examined be measuring the radiochemical yields of the radiopharmaceutical macroaggregates over time. The result are shown in Table 27, and demonstrate that these products are relatively stable over 16-72 hours:

TABLE 27
Stability of Radiopharmaceutical Macroaggregates Over Time

| | | | |
|---|---|---|---|
| Radionuclide | Fe | Ca | Gd |

Initial % yield

| | | | |
|---|---|---|---|
| ⁶⁷Ga citrate | 68.09 ± 1.14 | 13.84 ± 1.89 | 85.20 ± 2.00 |
| ²⁰¹Tl Cl | 90.71 ± 0.17 | 48.37 ± 0.38 | 44.09 ± 3.00 |
| ¹⁵³Sm EDTMP | 46.08 ± 4.11 | 76.40 ± 6.39 | 95.15 ± 3.29 |
| ⁸⁹Sr Cl | 80.58 ± 12.80 | 9.90 ± 1.73 | 12.08 ± 2.23 |
| ⁹⁰Y Cl | 27.8 ± 1.8 | 16.9 ± 14.5 | 86.3 ± 2.0 |
| ¹¹¹In Cl | 40.4 ± 1.8 | 69.1 ± 8.3 | 86.8 ± 6.1 |

% yield after suspension in PBS

| | | | |
|---|---|---|---|
| ⁶⁷Ga citrate (16 hours) | 57.15 ± 0.70 | | |
| ²⁰¹Tl Cl (16 hours) | 90.71 ± 0.17 | | |
| ¹⁵³Sm EDTMP | 41.90 ± 4.68 | 37.68 ± 5.84 | 59.10 ± 14.58 |
| (72 hours) | | | |
| ⁸⁹Sr Cl (72 hours) | 77.07 ± 12.59 | 5.43 ± 0.88 | 6.35 ± 1.16 |
| ⁹⁰Y Cl (24 hrs) | 17.3 ± 0.5 | 33.5 ± 3.4 | 59.5 ± 6.0 |
| ¹¹¹In Cl (24 Hrs) | 35.5 ± 3.4 | 60.5 ± 8.3 | 80.9 ± 9.5 |

To verify that the above radiopharmaceutical macroaggregates are generated by a mechanism likely related to adsorption, radiopharmaceutical macroaggregates generated using the above method were subjected to heating at 70-80° C. for 5 minutes, and then underwent 2 cycles of washings in PBS with centrifugation at 3000 rpm for 5 minutes. As shown in Table 28 below, the mechanism for generating these radiopharmaceutical macroaggregates is likely related to adsorption because significant dissociation occurred after the radiopharmaceutical macroaggregates were heated:

TABLE 28
Poor Stability of the Radiopharmaceutical Macroaggregates
with Heating

| | | | |
|---|---|---|---|
| Radionuclide | Fe | Ca | Gd |

Initial % yield

| | | | |
|---|---|---|---|
| ⁶⁷Ga citrate | 68.09 ± 1.14 | 13.84 ± 1.89 | 85.20 ± 2.00 |
| ²⁰¹Tl Cl | 90.71 ± 0.17 | 48.37 ± 0.38 | 44.09 ± 3.00 |
| ¹⁵³Sm EDTMP | 46.08 ± 4.11 | 76.40 ± 6.39 | 95.15 ± 3.29 |
| ⁸⁹Sr Cl | 80.58 ± 12.80 | 9.90 ± 1.73 | 12.08 ± 2.23 |

% yield after heating at 70-80° C. for 5 minutes

| | | | |
|---|---|---|---|
| ⁶⁷Ga citrate (16 hours) | 44.07 ± 4.61 | | |
| ²⁰¹Tl Cl (16 hours) | 85.25 ± 0.34 | | |
| ¹⁵³Sm EDTMP | 39.92 ± 4.95 | 36.04 ± 8.15 | 58.26 ± 13.26 |
| (72 hours) | | | |
| ⁸⁹Sr Cl (72 hours) | 72.41 ± 14.47 | 6.71 ± 1.21 | 5.16 ± 0.82 |

Finally, as shown in Table 29 below, radiopharmaceutical macroaggregates were generated with two different radionuclides by the mechanism of adsorption, demonstrating that these radiopharmaceutical macroaggregates can be generated using two or more radionuclides and a metal carrier. About 1-2 micro Ci of each isotope (⁶⁷Ga citrate or ²⁰¹Tl Cl) in approximately 50 μl was added to 1 mg of nonradioactive Fe macroaggregates. The nonradioactive Fe macroaggregates were prepared by adding NH₄OH to reach a pH of 7-13. About 1-2 micro Ci of each isotope (⁶⁷Ga citrate or ⁹⁰Y Cl) in approximately 50 μl was added to 1 mg of nonradioactive Gd macroaggregates. The nonradioactive Gd macroaggregates were prepared by adding NaOH to reach a pH of 7-13 The macroaggregates underwent 2 cycles of washing with PBS followed by centrifugation at 3000 rpm for 5 minutes. Radioactivities of the residuals were counted by the individual windows in a gamma counter and the individual component radioactivities were derived using Kramer's rule (standard solution for simultaneous linear equations, http://www.physicspost.com/articles.php?articleId=139, incorporated herein by reference).

TABLE 29
Double Labeling of Radiopharmaceutical Macroaggregates
by Adsorption
% yield of “single-radiolabel” Fe radiopharmaceutical
macroaggregates

| | |
|---|---|
| ⁶⁷Ga | 68.09 ± 1.14 |
| ²⁰¹Tl Cl | 90.71 ± 0.17 |

Individual % yield of “double-radiolabel” radiopharmaceutical
macroaggregates (⁶⁷Ga-²⁰¹Tl-Fe)

| | |
|---|---|
| ⁶⁷Ga | 64.08 |
| ²⁰¹Tl Cl | 87.53 |

% yield of “single-radiolabel” Gd radiopharmaceutical
macroaggregates

| | |
|---|---|
| ⁶⁷Ga | 85.20 ± 2.00 |
| ⁹⁰Y | 59.50 ± 6.0 |

Individual % yield of “double-radiolabel” radiopharmaceutical
macroaggregates (⁹⁰Y-⁶⁷Ga-Gd)

| | |
|---|---|
| ⁶⁷Ga | 62.02 |
| ⁹⁰Y | 48.23 |

Individual % yield of “double-radiolabel” radiopharmaceutical
macroaggregates (⁹⁰Y-⁶⁷Ga-Gd) at 24 hr indicating
stability of the radiopharmaceutical macroaggregates

| | |
|---|---|
| ⁶⁷Ga | 58.00 |
| ⁹⁰Y | 15.72 |

The above experiments indicate that only some radionuclides are able to label the nonradioactive particles tested through the mechanism of adsorption. Routine experimentation can be used to identify other combinations of radionuclides and nonradioactive particles that will generate radiopharmaceutical macroaggregates through the mechanism of adsorption. Interestingly this pattern of labeling using adsorption is different than when the same radionuclides and nonradioactive particles are co-precipitated to form radiopharmaceutical macroaggregates (see Example 1). Although radiopharmaceutical macroaggregates generated by the mechanism of adsorption may have lower labeling efficiencies and/or lower stability over time than those generated by co-precipitation, these radiopharmaceutical macroaggregates may be nevertheless clinically useful.

### EXAMPLE 5

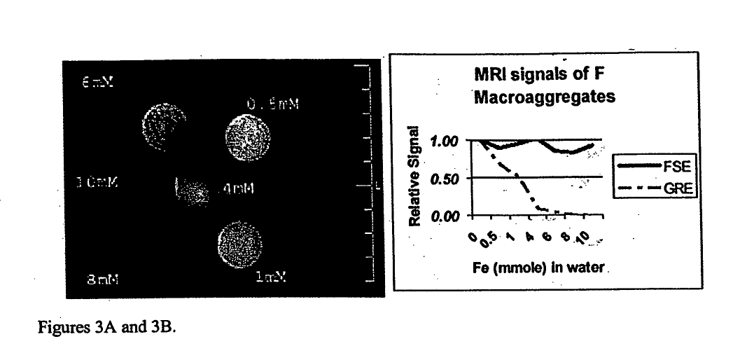

To demonstrate a paramagnetic radiopharmaceutical macroaggregate that provides magnetic signals for volumetric measurements and gamma rays for radioactivity measurements, Gallium-Iron macroaggregate (GIMA) was analyzed. GIMA provides paramagnetic signals for volume measurements by MR imaging while simultaneously emitting gamma rays for nuclear imaging. GIMA measures 10-30 micron in size by simple inspection under a microscope, and was used in human lung perfusion imaging until the advent of the current imaging agent of ^{99m}Tc-macroalbumin aggregates (Colombetti et al., J Nucl Med 11: 704-707,1970). An MRI study of the ⁶⁷Ga GIMA demonstrated decreases in Gradient Echo (GRE) signals as Fe contents increased to the concentration range intended for intratumoral injection (FIGS. 3A and 3B). In FIG. 3A, a GE Signa 1.5 T MRI scanner demonstrated decreasing GRE signals from 6 phantoms of 1 cc cylinders. In FIG. 3B, decreasing GRE signals with iron content was found with GRE pulse sequences but not with Fast Spin Echo (FSE) sequences.

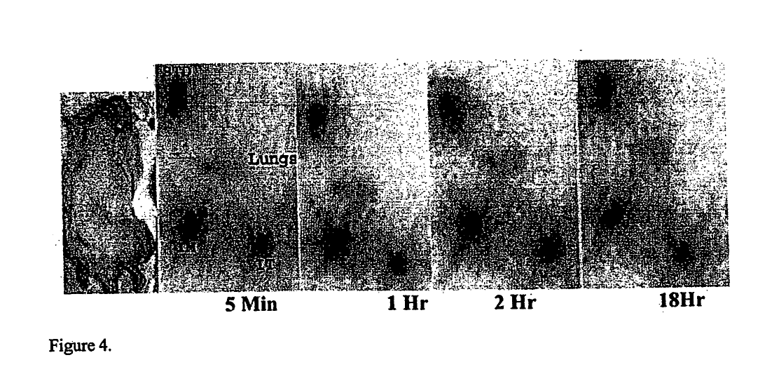

To make use of the physical half-life of 78 hours for ⁶⁷Ga, the ⁶⁷Ga/Fe macroaggregate was synthesized by methods disclosed herein to generate a ⁶⁷Ga GIMA with high specific activity. The high specific activity is due in part to the fact that no carrier-added ⁶⁷Ga citrate was used to produce the ⁶⁷Ga GIMA. 0.1 mCi ⁶⁷Ga GIMA was injected intratumorally (IT) and intramuscularly (IM) into the left leg of a 160 gram rat with a breast tumor implanted it in its right leg. As illustrated in FIG. 4, both intramuscular and intratumoral injection sites demonstrated prolonged retention of ⁶⁷Ga GIMA (65-80% at 18 hours). A ⁶⁷Ga standard was placed in the upper left corner of FIG. 4 as a control. Persistently low (<2%) lung uptake was also found in the rat, which may be related to leakage of ⁶⁷Ga GIMA into the systemic circulation during the IM injection.

### EXAMPLE 6

One potential utility of a paramagnetic radiopharmaceutical macroaggregate is suppression of in vivo tumor growth. This utility was demonstrated using the paramagnetic radiopharmaceutical macroaggregate ⁶⁷Ga GIMA. On day zero, 100,000 rat mammary cancer 13762F tumor cells were implanted in a volume of 0.15 ml into the right thigh muscle of a Fischer 344 female rat weighing approximately 160 grams. In one set of experiments, on day 10 the rats injected with tumor cells were subsequent treated with 0.2 or 0.8 mCi of ⁶⁷Ga GIMA (0.2 mCi ⁶⁷Ga, 1 mg Fe, and 0.8 mCi ⁶⁷Ga, 1 mg Fe respectively). The ⁶⁷Ga GIMA was injected intratumorally in a volume of 0.2 ml on day 10 after the tumors became palpable in the rats. In another set of experiments, on day 3, 1 mCi of ⁶⁷Ga GIMA (1 mCi ⁶⁷Ga, 1 mg Fe) in 0.3 ml was injected intramuscularly into the same location of the right thigh of a set of the rats injected with tumor cells. The remaining rats injected with tumor cells were used as controls. Tumor sizes were then monitored regularly.

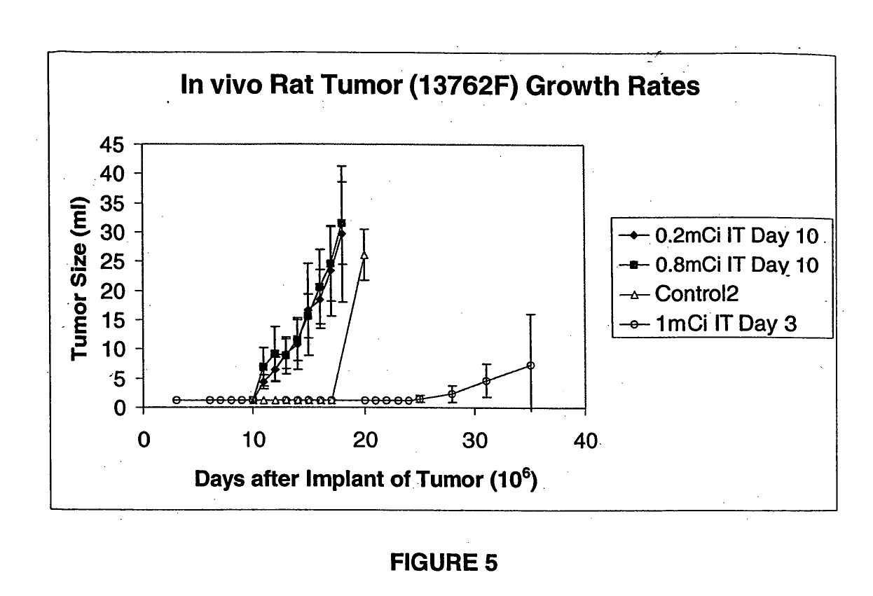

As shown in FIG. 5, tumor development was not reduced in rats treated with 0.2 or 0.8 mCi of ⁶⁷Ga GIMA on day 10 as compared to the control. The parallel control group developed tumors at a slightly later time than those of treated with 0.2 or 0.8 mCi of ⁶⁷Ga GIMA, but the tumors in the control group were definitely much more aggressive than the tumors in the rats treated with ⁶⁷Ga GIMA. It is uncertain why these dosages of ⁶⁷Ga GIMA were not effective for suppressing the rate of tumor growth in these rats. Possibly the rats were simply treated too late with ⁶⁷Ga GIMA, or the radioactivity levels of the ⁶⁷Ga GIMA were not sufficient to suppress tumor growth. For example, day 10 tumors, which have a more heterogeneous architecture and cell distribution, may require greater radiation to destroy. In the set of rats treated with 1 mCi of ⁶⁷Ga GIMA beginning on day 3, FIG. 5 shows that the rate of tumor growth in these rats was significantly reduced as compared to the control. This demonstrates that injection of the paramagnetic radiopharmaceutical macroaggregate ⁶⁷Ga GIMA is able to suppress tumor growth in vivo. Repeated in vivo rat experiments confirmed tumor suppression by GIMA prepared with co-precipitation, GIMA prepared by adsorption, and ⁹⁰Y iron macroaggregates (YIMA) prepared by co-precipitation.

Dosimetry of the injected ⁶⁷Ga GIMA can also be estimated using the dosimetry simulations developed by one of the inventors disclosed herein, as shown in FIGS. 1 and 2. For example, in the above experiment 1 mCi of ⁶⁷Ga GIMA was injected in 0.2 ml was found to have a distributed volume of 0.5 cc 1 hour after injection. At least 90% of the ⁶⁷Ga was also found to have been retained in the injected area after 35 days. Therefore, using the simulated radiation absorbed dose from the curves of ⁶⁷Ga at 0.4 cc in FIG. 1, the radiation absorbed can be grossly estimated as: S value (cGy/mCi-Hr)×residence time (Hr)×radioactivity (mCi)=201×(1.4×0.9×78 Hr)×1=19750 cGy or 198 Gy. Based on these values it is not surprising that tumor growth was suppressed in these rats. From FIG. 2 the 10% isodose range can be calculated to be about 0.02 cm. Escape from tumor suppression may be related to the short range of the injected ⁶⁷Ga GIMA, which has approximately a 0.5 cc distributed volume and a 10% isodose range of 0.02 cm, because technically it is very difficult to subsequently inject the ⁶⁷Ga GIMA within mm of the identical location the tumor cells were initially injected into.

### EXAMPLE 7

Clinical trials have confirmed the usefulness of sealed radionuclides as internal radiation sources. To confirm the feasibility of using intratumoral injection of unsealed radionuclide an as internal radiation source, the paramagnetic radiopharmaceutical GIMA is used to evaluate intratumoral injection as an alternative method to effectively ablate solid tumors while sparing normal tissues. To evaluate GIMA, the human breast tumor model system is used to measure the spatial and temporal distribution of injected GIMA. After GIMA is injected intratumorally it will disperse in the tumor, but will remain contained within the tumor, leading to high absorption of radiation within the tumor from GIMA, but low absorption in surrounding tissues and organs.

Patients are recruited from female breast cancer patients scheduled for surgery at least one-week after the planned day of injection. One of the inclusion criteria is a tumor size of 2-3 cm or 4-15 cc in volume. No spillage outside of the tumor is expected from an injection of 1 cc. The radiopharmaceuticals ⁶⁸Ga GIMA and ⁶⁷Ga GIMA are synthesized under sterile conditions and tested for pyrogenicity using the LAL test (Whittaker Inc., Walkersville, Md.) before use. A total of 15 patients in 3 groups of 5 patients each are studied. All patients are recruited under an IRB approved protocol with informed consent obtained. The patients are injected with GIMA intratumorally to measure the radiation dosimetry for GIMA. To aid the measurements of radiation dosimetry, MR imaging and PET or high-resolution scintigrams are used to generate accurate measurements of the spatial and temporal profiles required for radiation absorbed dose estimates at the injection site, surrounding breast tissues, and vital organs. The MRI and nuclear imaging studies follow routine clinical procedure.

All 15 patients undergo MRI studies daily for a total of 5 consecutive days. The first group of 5 patients receives 0.5 mCi of ⁶⁸Ga GIMA (in 1 cc saline) intratumorally under MRI guidance and then undergo PET studies on the first day. A second group received 0.2 mCi intratumoral injections of ⁶⁷Ga GIMA (in 1 cc saline) under MRI and undergo whole-body and chest scintigraphy at 2, 4, 6 hours and 2, 3, 4, and 5 days. The patients are scanned using a GE Signa Lx 1.5 Tesla MRI scanner equipped with a high performance gradient system (amplitude=22 mT/m; slew rate=120 T/m/s). A phased-array bilateral breast RF coil is used to maximize the signal-to-noise ratio. A breast positioning system with two compression plates is used to hold the breast in a reproducible location, thereby maximizing the chance images from different scan days will register. The gross distribution of the composition was also monitored by ultrasonography.

To inject the GIMA intratumorally, the breast tumor in a patient is first localized using a fast T1-weighted 3D pulse sequence. If necessary, Gd-DTPA contrast agent is administered intravenously to assist in identifying the lesion. An MR-compatible disposable sterile needle is placed intra-tumorally, carefully avoiding any areas of necrosis. An MR scan is performed to ensure the proper location of the needles. Prior to injection, a high-resolution baseline image is obtained using a gradient echo (GRE) pulse sequence with parameters selected to be sensitive to T2*. The GIMA is injected into the tumor over 1 minute, and the needle is then slowly removed. This procedure is similar to routine breast lymphoscintigraphy (Johnson et al., Am J Surg 179:386-88, 2000, Doting et al., Cancer 88:2546-52, 2000). Immediately after injection, images from two sessions of multi-phase T2*-weighted MRI are acquired using the same pulse sequence in quick successions for the next 60 minutes. The volume of the injectate is determined from the voxels with >10% decrease in the T2* signal compared to earlier images. If subsequent scintigraphy/PET detects movement of GIMA, the area of the lymph drainage is included for similar analysis. Serial MRI are performed daily for the next 4 days to determine the geometry and distribution of GIMA.

The early phase of GIMA movement is studied with ⁶⁸Ga GIMA and PET in the first group of 5 patients because accurate localization and quantitation of radioactivity are derived from the superior accuracy and resolution of PET. However, delayed PET studies after the first day are not useful because ⁶⁸Ga decays rapidly (1.2 hour half-life). Therefore, the second and third groups of patients receive ⁶⁷Ga GIMA to assess the later phase (2-4 days) of radioactivity movements. After MR guided injection and imaging, the patient is sent to the Nuclear Medicine/PET Clinic in a gurney to minimize extraneous motion of the breast. The radioactivity residence time in the tumor and lymph nodes is derived from either serial scintigrams or serial PET. Patients injected with ⁶⁸Ga GIMA undergo PET (1, 2, 3 and 4 hours) from the neck down to the pelvis with attenuation correction using a high resolution Siemens HR Plus PET or a GE PET/CT scanner. Images are then reconstructed in 3-D mode and the ⁶⁸Ga voxel concentration in the tumor, lymph node, and/or normal organs is measured. For patients with ⁶⁷Ga GIMA, scintigrams are acquired in a Siemens dual-head ECAM gamma camera equipped with ultra-high resolution collimator. This combination is able to achieve a system resolution of 7 mm FWHM with ^{99m}Tc at a distance of 10 cm. One initial transmission scan is performed, and then whole-body and planar imaging continues at 2, 4, 6 hours and 2, 3, 4, and 5 days after injection. Alternatively, ^{99m}TcO₄-labeled Fe aggregates (e.g., Table 2 with a 0.94 radiochemical yield), are used to study the short-term (1-8 hours) biodistribution in humans because of the 6-hour physical half-life of ^{99m}TcO₄ and because ^{99m}TcO₄ provides ideal imaging characteristics for gamma cameras.

After the above measurements are completed, histologic changes from radiation effects (McCormick et al., Radiation Therapy Oncology Group. Research Plan 2002-2006. Breast Cancer Working Group. Int J of Radiation Concol, Bilol, Physics. 51(3Suppl 2):56-7, 2001; Mirza et al., Cancer J 7:95-102, 2001, incorporated herein by reference) in and around the tumor/lymph nodes are evaluated and correlated with predicted and measured dosimetry. Selected histopathologic slices from patients injected with ⁶⁷Ga GIMA are temporarily secured for autoradiography to visualize geographic distribution. The GIMA distribution from autoradiography is also used to correlate MRI-derived volumetric data with radioactivity data from nuclear imaging. Another analogous clinical trial will study radiation dosimetry of GIMA in human prostate cancer to derive the biodistribution or segregation of particulate radiopharmaceuticals in human cancers/organs. The dosimetry derived will allow the use of either ⁶⁷Ga or other radionuclides (including ⁹⁰Y) to produce particulate radiopharmaceuticals for human cancer therapies.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A radiopharmaceutical macroaggregate composition for the treatment of abnormal tissue comprising particles having a minimum size of one micron, wherein the particles comprise a metal and one or more radioactive isotopes, and have sufficient radioactivity for locoregional ablation of cells in the abnormal tissue.

2. The composition of claim 1, wherein the radiopharmaceutical macroaggregate composition is paramagnetic.

3. The composition of claim 1, wherein the radiopharmaceutical macroaggregate composition is nonparamagnetic.

4. The composition of claim 1, wherein the metal is iron or gadolinium.

5. The composition of claim 1, wherein the metal is calcium.

6. The composition of claim 1, wherein the one or more radioactive isotopes are selected from the group consisting of Gallium-67 (⁶⁷Ga), Yttrium-90 (⁹⁰Y), Yttrium-86 (⁸⁶Y), Gallium-68 (⁶⁸Ga), Thallium-201 (²⁰¹Tl), Strontium-89 (⁸⁹Sr), Indium-111 (¹¹¹In), Iodine-131 (¹³¹I, Samarium-153 (¹⁵³Sm), Technetium-99m (^{99m}Tc), Rhenium-186 (¹⁸⁶Re), Rhenium-188 (¹⁸⁸Re), Copper-62 (⁶²Cu), and Copper-64 (⁶⁴Cu).

7. The composition of claim 1, wherein the radiopharmaceutical macroaggregate composition comprises particulates or microspheres.

8. The composition of claim 7, wherein the particulates or microspheres comprise glass.

9. The composition of claim 7, wherein the particulates or microspheres comprise ceramic.

10. The composition of claim 1, wherein the one or more radioactive isotopes emit beta radiations or positrons.

11. The composition of claim 1, wherein the particles comprise a metal and one radioactive isotope.

12. The composition of claim 11, wherein the radioactive isotope is a cation.

13. The composition of claim 11, wherein the radioactive isotope is an anion.

14. The composition of claim 1, wherein the particles comprise a metal and two radioactive isotopes.

15. The composition of claim 14, wherein one or both of the radioactive isotope are cations.

16. The composition of claim 14, wherein one or both of the radioactive isotope are anions.

17. The composition of claim 14, wherein one of the radioactive isotopes is Holmium-166 (¹⁶⁶Ho).

18. The composition of claim 1, wherein the particles further comprise Phytate.

19. The composition of claim 10, wherein the particles further comprise Phytate.

20. The composition of claim 14, wherein the particles further comprise Phytate.

21. The composition of claim 1, wherein the ratio of radioactive isotopes to metal is about 106:1.

22. The composition of claim 1, wherein the particles are biodegradable.

23. The composition of claim 1, wherein the size of the particles is from about 5 to about 50 microns.

24. A method for the locoregional treatment of abnormal tissue, comprising administering a radiopharmaceutical macroaggregate composition to a subject in the region of the abnormal tissue, wherein the radiopharmaceutical macroaggregate composition comprises particles having a minimum size of one micron, wherein the particles comprise a metal and one or more radioactive isotopes, and have an effective amount of radioactivity for locoregional ablation of cells in the abnormal tissue.

25. The method of claim 24, wherein the subject is human.

26. The method of claim 24, wherein the one or more radioactive isotopes are selected from the group consisting of Gallium-67 (⁶⁷Ga), Yttrium-90 (⁹⁰Y), Yttrium-86 (⁸⁶Y), Gallium-68 (⁶⁸Ga), Thallium-201 (²⁰¹Tl), Strontium-89 (⁸⁹Sr), Indium-111 (¹¹¹In), Iodine-131 (¹³¹I), Samarium-153 (¹⁵³Sm), Holmium-166 (1⁶⁶Ho), Technetium-99m (^{99m}Tc), Rhenium-186 (¹⁸⁶Re), Rhenium-188 (¹⁸⁸Re), Copper-62 (⁶²Cu), and Copper-64 (⁶⁴Cu).

27. The method of claim 24, wherein the metal is iron.

28. The method of claim 24, wherein the metal is gadolinium.

29. The method of claim 28, wherein the radiopharmaceutical macroaggregate composition comprising gadolinium is exposed to neutron irradiation for the locoregional treatment of abnormal tissue.

30. The method of claim 24, wherein the radiopharmaceutical macroaggregate composition is used in Selective Internal Radiation Therapy (SIRT).

31. The method of claim 24, wherein the radiopharmaceutical macroaggregate composition is paramagnetic.

32. The method of claim 31, wherein the paramagnetic properties of the radiopharmaceutical macroaggregate composition are used to measure the geographic distribution and derive radiation dosimetry of the radioactive composition.

33. The method of claim 31, wherein the paramagnetic properties of the radiopharmaceutical macroaggregate composition is used for local hyperthermia therapy.

34. The method of claim 31, wherein magnetic resonance imaging (MRI), Positron Emission Tomography (PET), ultrasonography, or high resolution gamma scintigraphy is used to measure the spatial and temporal profiles of the radioactive composition.

35. The method of claim 31, wherein a Computed Tomography (CT) scanner is used to localize the radioactive composition.

36. The method of claim 24, wherein the metal is calcium.

37. The method of claim 36, wherein a Computed Tomography (CT) scanner or ultrasonography is used to localize the radioactive composition.

38. The method of claim 24, wherein the particles further comprise Phytate.

39. The method of claim 24, wherein the radiopharmaceutical macroaggregate composition is administered by injection.

40. The method of claim 39, wherein the injection is intratumoral, intravenous, intravascular, intraparenchymal, intraarterial, intracavitary, intra-pleural, intraperitonal, or intrathecal.

41. The method of claim 24, wherein the abnormal tissue is a neoplasm.

42. The method of claim 41, wherein the neoplasm is a tumor.

43. The method of claim 42, wherein the radiopharmaceutical macroaggregate composition is administered directly into the tumor by injection.

44. The method of claim 24, wherein the abnormal tissue is synovial tissue.

45. A radiopharmaceutical macroaggregate composition for the treatment of abnormal tissue comprising particles having a minimum size of one micron, wherein the particles comprise a metal and one or more radioactive isotopes, and have sufficient radioactivity for locoregional ablation of cells in the abnormal, produced by a process comprising the steps of:
(a) mixing one or more radioactive isotopes with a metal chloride;
(b) adding an alkaline to the mixture of part a to precipitate the radioactive isotopes with the metal to form the particles;
(c) separating the precipitated particles from any remaining soluble radioactive isotopes from the particles; and
(d) isolating the radioactive particles.

46. The process of claim 45, wherein the metal chloride is selected from the group consisting of ferric chloride (FeCl₃), calcium chloride (CaCl₂), and gandolinium chloride (GdCl₃).

47. The process of claim 45, wherein the alkaline is sodium hydroxide or ammonium hydroxide.

48. A radiopharmaceutical macroaggregate composition for the treatment of abnormal tissue comprising particles having a minimum size of one micron, wherein the particles comprise a metal and one or more radioactive isotopes, and have sufficient radioactivity for locoregional ablation of cells in the abnormal, produced by a process comprising the steps of:
(a) adding an alkaline to a metal chloride to form a precipitate;
(b) mixing one or more radioactive isotopes with the precipitate of part (a) to allow the radioactive isotopes to adsorb to the precipitate and generate a radioactive precipitate;
(c) separating the radioactive precipitate of part (b) from any remaining soluble radioactive isotopes; and
(d) isolating the radioactive precipitate.

49. The process of claim 48, wherein the metal chloride is selected from the group consisting of ferric chloride (FeCl₃), calcium chloride (CaCl₂), and gandolinium chloride (GdCl₃).

50. The process of claim 48, wherein the alkaline is sodium hydroxide or ammonium hydroxide.

51. The process of claim 48, wherein the radioactive precipitate of part (b) is separated from any remaining soluble radioactive isotopes by centrifugation.

52. A method of acupuncture therapy for an acupuncture-responsive condition, comprising administering a radiopharmaceutical macroaggregate composition into one or more acupuncture points of a subject, wherein the radiopharmaceutical macroaggregate composition comprises particles having a minimum size of one micron, wherein the particles comprise a metal and one or more radioactive isotopes, and have an effective amount of radioactivity to enhance the acupuncture therapy.
